(19) United States
(12) Patent Application Publication
(10) Pub. No.: US 2024/0006075 A1
(43) Pub. Date: Jan. 4, 2024

(54) SYSTEMS AND METHODS FOR PREDICTING AND DETECTING POST-OPERATIVE COMPLICATIONS

(71) Applicant: NERv Technology Inc., Kitchener (CA)

(72) Inventors: Nour Hesham Atif Ahmed Helwa, Waterloo (CA); Abdallah Hassen El-Falou, Kitchener (CA); Ricky Tjandra, Kitchener (CA); Khaled Berry, Oakville (CA); William Michael Kovacs Pitman, Kitchener (CA)

(21) Appl. No.: 18/344,258

(22) Filed: Jun. 29, 2023

Related U.S. Application Data
(60) Provisional application No. 63/357,370, filed on Jun. 30, 2022.

(51) Int. Cl. G16H 50/30; G16H 10/60; G16H 50/70

(52) U.S. Cl. CPC G16H 50/30; G16H 10/60; G16H 50/70

(57) ABSTRACT

Disclosed herein are computer-implemented methods and systems for assessing a risk value of a target patient, the method comprising: receiving at a server, via a connection mechanism, target patient data; estimating at the server, using one or more risk assessment models, one or more risk values associated with the target patient data. Target patient data may comprise historical data, patient population-level data, and real-time data. Risk assessment models may comprise one or more of: historical data risk assessment models, real-time data risk assessment models, or a combined risk assessment model. Methods for training such models are described. Systems and methods as described preferably enable continuous patient monitoring based on a patient's historical data and continuous, real-time physiological data.

### CROSS-REFERENCE TO RELATED APPLICATIONS

This application claims priority from U.S. Provisional Patent Application No. 63/357,370, filed on Jun. 30, 2022, the disclosure of which is hereby incorporated herein in its entirety by reference.

### FIELD OF INVENTION

The present disclosure relates to systems, methods and devices for monitoring, predicting and detecting different forms of post-operative complications. The present invention relates to patient monitoring and risk assessment, and more specifically, to computer implemented systems and methods for monitoring and assessing patient risk, particularly post-surgery.

### BACKGROUND

Surgical procedures may use open and minimally invasive techniques on users, such as patients, in order to identify and treat pathological conditions or improve body functions. Surgeries performed due to a variety of reasons have an inherent risk of post-operative complications such as hemorrhages, infections and leakages to develop.

One of the most dangerous complications for surgery is a complication known as anastomotic leakage. Anastomotic leakage may develop after an anastomosis is performed where two organs are surgically connected, and is most commonly observed in gastrointestinal surgery. Anastomotic leakage leads to luminal contents leaking into the peritoneal cavity which may cause a cascade of deadly complications to arise. This typically involves a form of severe sepsis, peritonitis, morbidity and it may lead to mortality.

Using traditional techniques, it can take three to seven days on average for a leak to be diagnosed. This is very dangerous especially considering that every hour of delay causes a considerable increase in the morbidity and mortality risk for the patient.

Each year, 70 million major abdominal surgery (MAS) procedures are performed globally. MAS includes pancreatic, hepatobiliary, and colorectal surgeries with a primary anastomosis. These surgeries have a complication rate of 30-60%, 20% of which have significant detrimental effects that require invasive treatments and enhanced patient monitoring. The occurrence of such complications can have dire consequences, both acute and long-term, including a significant degree of morbidity and mortality for affected patients.

Post surgical complications may further comprise, without limitation, hemorrhage, post-operative leakages, ischemia, infection, and sepsis.

Typically, medical facilities wait for clinical factors such as abdominal pain, fever and tachycardia to arise before diagnosis. Existing technologies for detection of post-operative complications, such as anastomotic leakage, may be nonspecific, inefficient, time-consuming, expensive, and/or lacking in the ability to provide real-time detection of the complication.

Additionally, even though there is a plethora of patient data available, current technologies are unable to provide care-providers with an accurate prediction of whether patients will develop post surgical complications.

Current patient monitoring systems may not be informed by historical data, often relying on physician assessments, which may be prone to bias. They may not be configured to receive continuous real-time data from a patient, such as bio-signal data, or post-operative data (including, but not limited to, vitals measurements).

Alternatively, patient monitoring systems in the prior art may rely on static models, which may not be continually updated based on newly available patient data.

Such models are typically trained on data that may have missing values. Common methods to overcome missing data issues include imputation of missing values or removing entries with missing values from the dataset entirely.

Removing patients with missing values limits the available number of training patients, and imputing can be problematic if the number of imputed values is large compared to the number of available values and can be non-trivial for full collections of sensor measurements.

US20220361823 discloses wearable blood pressure biosensors, systems, and methods for short term blood pressure predictions of average blood pressures for future time periods. The system can include models trained to predict future systolic values, diastolic blood pressure values, and/or trends. The models can be trained on data related to the personalized body characteristics of the user. However, the models do not receive data from a plurality of sensors receiving data from multiple patients—rather, they are individualized for a user and therefore the models are developed from scratch with every new user.

U.S. Ser. No. 10/463,312 Discloses embodiments of methods and systems for predicting mortality of a patient. The method comprises categorizing a historical data into a first category and a second category. The method further comprises determining a first test parameter and a second test parameter based on at least one of a sample data of a first patient and the historical data corresponding to at least one of the first category and the second category. The method further comprises determining a probability score based on a cumulative distribution of at least one of the first test parameter and the second test parameter. The method further comprises categorizing the sample data in one of the first category and the second category based on the probability score. Further, the method comprises predicting the mortality of the first patient based on at least the categorization of the sample data of the first patient. The mortality prediction method is limited in that it does not associate biological data with a given time stamp, and, by nature, cannot provide real-time predictions. It is configured to predict mortality of a patient based on static, measured biological data, and does not associate the time at which the data is measured with a probability of mortality, since the model is trained on static physiological conditions.

US20220238235 discloses a computer-implemented method for providing outcome tracking of patients, which may include generating an event trigger for the patient, wherein during a duration of the patient's recovery, the event trigger may correspond to values of a patient biomarker over or under threshold values while the patient is performing a post-surgery activity related to the patient's recovery. The computer-implemented method may include receiving actual patient biomarker data from a patient sensor system for the patient while the patient is performing a post-surgery activity. If the actual patient biomarker data includes values over or under the threshold value while the patient is performing a post-surgery activity, the method may include triggering the event trigger. The method may include generating a notification alert corresponding to the event trigger. Outside of a hospital, patient sensor systems comprise medical wearables, such as watches and headbands, meaning that the method is not configured to receive sensor information after a surgery related to continuously flowing biofluids of the patient, but rather related to externally measured data such as heart rate. In this sense it may monitor how the patient is recovering based on these external factors, but may not determine post operative risk specific to a surgery based on continuous measurement of biomarkers from an inline device, such as leakage or infection.

A new method and system have been developed to gather continuous biomarker data from patients in order to detect postoperative complications earlier compared to the standard of care (SOC). This system includes a novel method that continuously calculates a probability of a particular patient's risk of developing a post operative complication at that particular point in time. This allows healthcare providers to detect post operative complications earlier than SOC, increasing patient outcomes.

Despite the major advances in postoperative care and the development of numerous novel monitoring techniques, to date, there are no devices or technologies specifically de signed for the early detection or prediction of the occurrence of postoperative complications following a surgery. Therefore, there may be a need for systems and methods which may perform continuous patient monitoring and risk assessment, using patients' historical data, real-time data, or a combination of both.

### BRIEF SUMMARY

The following presents a simplified summary of the general inventive concept(s) described herein to provide a basic understanding of some aspects of the disclosure. This summary is not an extensive overview of the disclosure. It is not intended to restrict key or critical elements of the embodiments of the disclosure or to delineate their scope beyond that which is explicitly or implicitly described by the following description and claims.

Leak incidence rate from surgical procedures can vary from 1% to 40% in some cases. Causes behind the development of anastomotic leaks are still being studied with no definitive causes identified yet. There are however risk factors that are associated with higher incidence rate such as but not limited to age, gender, organ tension, local ischemia, medical history, and surgical errors.

Physiological changes, such as tissue ischemia followed by necrosis, may occur before and during the development of an anastomotic leak. These changes may present themselves as subtle changes in the patient's vital parameters that may not be detected by the current standard of care.

It is an object of the invention to provide systems and methods for predicting and detecting postoperative complications.

The present disclosure provides systems, methods and devices for analyzing bodily and luminal fluids, which includes, without limitation, peritoneal fluid, peritoneal drainage fluid, pleural drainage fluid, gastric juice, fecal matter, bile fluid, urine, amniotic fluid, dialysate, sebum, or blood. The fluid may be continuously monitored for changes and trends in specific analytes and biological properties. Examples of these properties and analytes include, but are not limited to, pH, lactate, electrolytes, impedance, conductivity, dissolved oxygen, dissolved CO2, temperature, inflammatory markers, enzymes, bacterial proteins, RNA or lipids. Systems, methods and devices disclosed herein may be used for various diagnostic applications such as, but not limited to, post-operative leakages, ischemia, infection, and sepsis.

In some embodiments, sensors such as biosensors may be placed on a catheter, and the catheter may be inserted into the body and may allow fluid to be injected into or withdrawn from the body. The catheter can be placed proximal to the surgical site in order to monitor the milieu of the biological fluid proximate to the region. The fluid can be directly sensed locally without the need for negative pressure or it can use negative pressure to assist the fluid to be driven through the catheter. Any number of sensors can be placed on the surface of the catheters such that they are directly in contact with the biological fluid surrounding the area of interest such as the suture line in the case of an anastomosis for example. Sensors may also be placed on the inside of the catheter, a balloon, a pump or any tubing where the fluid can be collected.

In a further embodiment, sensors may be housed within a system that can be placed inline with a catheter. The catheter can be placed proximal to the surgical site in order to monitor the milieu of the peritoneal fluid proximate to the region. The system may be an extension of an existing catheter system. The system may be attached at any time when the catheter is being placed or at a later date.

Conveniently, early management to handle complications and address leakages, using techniques disclosed herein, may significantly mitigate risks associated with such complications.

Existing techniques for handling complications may involve using interventional radiology techniques to handle existing complications with patients. In the case of leakages this may involve techniques such as placing in drains, placing stents, enforcing the staple line sutures. These interventions may be done endoscopically without the need for a second surgery to be performed. Monitoring the user status using systems and techniques as disclosed herein may allow for a more effective treatment plan and earlier intervention if the complication appears again.

In addition, techniques disclosed herein may allow for home monitoring of the post-operative journey as more patients are moved to out-patient monitoring settings. Furthermore, techniques disclosed herein may allow for users to continuously monitor the status of a patient-surgery, which may be an improvement over existing diagnostic tests that take a sample at a specific point in time, which may not be indicative of a patient's status.

In accordance with an aspect of the invention there is provided a computer-implemented method for monitoring drainage fluid from a patient, the method comprising: receiving biosignal data from one or more biosensors coupled to the patient, said sensors in fluid communication with the drainage fluid and storing the pH data in a memory; calculating a cumulative average value of the percentage of data points below a predetermined baseline threshold value for a current post-operative period; marking current post-operative period as “High Risk” if the cumulative average value exceeds the predetermined baseline threshold value, otherwise, indicating as “Low Risk”.

In accordance with another aspect of the invention there is provided a system for monitoring a user, the system comprising: a processor; a memory in communication with the processor, the memory storing instructions that, when executed by the processor cause the processor to perform the method as described above.

According to an aspect, there is provided a monitoring device comprising: an input port attachable for fluid communication with a catheter, the catheter for insertion in a body of a user, for receiving fluid from the body of the user; an output port, generally parallel to the input port, in fluid communication with a fluid reservoir; a fluid channel defining fluid communication between the input port and the output port; and a biosensor, in communication with a computing device, for continuously measuring bio signal data of the fluid in the fluid channel, the biosensor including an electrode pair.

In some embodiments, the computing device is for determining a condition of the user based at least in part on the bio-signal data.

In some embodiments, the biosensor includes an impedance sensor for detecting a conductivity of the fluid in the fluid channel.

In some embodiments, the biosensor includes a pH sensor for detecting a pH level in the fluid in the fluid channel.

In some embodiments, the biosensor includes at least one of a lactate sensor, an amylase sensor, a urea sensor, or a creatinine sensor.

In some embodiments, the device further comprises a flow sensor for continuously determining a flow rate of the fluid in the fluid channel over time.

In some embodiments, the device further comprises a light-based sensor including a light transmitter and a light receiver for detecting transmission of light through the fluid in the fluid channel.

In some embodiments, the light-based sensor is configured to detect a color of the fluid based at least in part on a detected wavelength.

In some embodiments, the device further comprises a temperature sensor for detecting a temperature of the fluid in the fluid channel.

In some embodiments, the biosensor is disposed on a substrate in fluid communication with the fluid channel.

In some embodiments, the electrode pair is disposed sequentially along a length of the fluid channel.

According to another aspect, there is provided a computer-implemented method for monitoring a user, the method comprising: receiving bio-signal data continuously from a biosensor in fluid communication with the fluid; determining a condition of the user based at least in part on the bio-signal data; and predicting a future occurrence of a complication based at least in part on the condition of the user.

In some embodiments, the method further comprises receiving a profile of the user, the profile of the user including information related to a surgical procedure performed on the user, wherein the future occurrence of the complication is predicted based at least in part on the profile of the user.

In some embodiments, the method further comprises updating the profile of the user based at least in part on the bio-signal data.

In some embodiments, the method further comprises receiving flow data continuously from a flow sensor in fluid communication with fluid from a body of a user; and determining, based at least in part on the flow data, a rate of flow of the fluid, wherein the condition of the user is determined based at least in part on the rate of flow.

In some embodiments, the method further comprises determining a change in the rate of flow of the fluid over time and a change in bio-signal data over time, and the predicting the future occurrence is based at least in part on the change in the rate of flow and the change in bio-signal data.

In some embodiments, the flow data is received in near real-time.

In some embodiments, the bio-signal data is received in near real-time.

In some embodiments, the method further comprises receiving light data associated with transmission of light through the fluid from a light-based sensor in fluid communication with the fluid.

In some embodiments, the method further comprises determining a color of the fluid based at least in part on the light data.

In some embodiments, the method further comprises receiving temperature data of the fluid from a temperature sensor in fluid communication with the fluid. In some embodiments, the method further comprises modulating the bio signal data based at least in part on the temperature data.

In some embodiments, the method further comprises determining a risk factor of the user based on a cross-correlation with a trend of bio-signal data of other users.

In some embodiments, the condition of the user is based at least in part on determining whether the bio-signal data is within bounds of a threshold.

According to a further aspect, there is provided a system for monitoring a user, comprising: a processor; a memory in communication with the processor, the memory storing instructions that, when executed by the processor cause the processor to perform a method as described herein.

Current SOC relies on a combination of standardized management guidelines and individual experiences of healthcare providers. There is active research on the use of various risk factors and novel biomarkers for early detection of post operative complications.

Embodiments of the present invention include methods to calculate continuous, real-time numeric score based on pH of drainage fluid that is related to the probability of patients developing post-operative complications. These methods are novel and do not exist in the current SOC. The methods allow healthcare providers (HCP) to get a real-time status of their patients for closer monitoring, intervention or discharge earlier compared to the SOC.

The methods can integrate more than one biomarker data or other sources data that is available to the HCP (e.g., EMR/EHR, other sensors or medical devices) to give a more accurate probability score.

The methods can be deployed in firmware or cloud and updated/improved as needed, or when newly acquired data is available.

The methods can be adapted to detect different types of post operative complications.

The methods can be used with pH data from any source or device, including a continuous monitoring device or a point measurement from a benchtop pH meter.

The methods may further be used with other biomarker or biosignal data from any source or device, including inline and/or continuous monitoring devices.

According to an aspect of the invention, there is disclosed a computer-implemented method for assessing a risk value of a target patient, the method comprising: receiving at a server, via a connection mechanism, target patient data; estimating at the server, using one or more risk assessment models, one or more risk values associated with the target patient data.

In an embodiment, the target patient data comprises one or more of: historical data, patient population-level data, and real-time data.

In one embodiment, the one or more risk assessment models comprise one or more of: a historical data risk assessment model, a real-time data risk assessment model, and a combined risk assessment model, the combined risk assessment model comprising one or more risk assessment models.

In one embodiment, the historical data risk assessment model is trained by: receiving, at a server comprising one or more processors and a memory, historical data corresponding to a plurality of patients, the one or more processors comprising one or more of a mapping engine and a standardization engine, the historical data for each patient of the plurality of patients corresponding to an indication of whether a patient encountered a complication; generating pre-processed historical data by: mapping, via the mapping engine, the historical data for each patient of the plurality of patients, to numerical values; standardizing, via the standardization engine, historical data for each patient of the plurality of patients; performing a regression analysis on the pre-processed data, the regression analysis determining a relationship between historical data and a risk value.

In one embodiment, real-time data risk assessment model is trained by: receiving, at a server comprising one or more processors and a memory, time-marked data corresponding to signal data measured by a plurality of sensors coupled to a plurality of patients, the one or more processors comprising one or more of a filtering and augmentation engine and a standardization engine, the time-marked data for each patient of the plurality of patients corresponding to an indication of whether a patient encountered a complication; generating pre-processed time-marked data by: filtering and augmenting, via the filtering and augmentation engine, the time-marked data for each patient of the plurality of patients; standardizing, via the standardization engine, the time-marked data for each patient of the plurality of patients; performing one or more regression analyses on the pre-processed time-marked data, the one or more regression analyses determining one or more relationships between real-time data and a risk value.

According to an embodiment of the invention, the historical data comprises one or more of the target patient's, or target patient population's: pre-operative risk factors, medical records, surgical history, individual health indicators, and surgical parameters.

According to an embodiment of the invention, the real-time data comprises sensor data from one or more sensors continuously measuring signals associated with a physiological condition of the target patient.

In an embodiment, the method further comprises notifying a user of said risk value.

In an embodiment, the risk value is estimated continuously.

According to an embodiment of the invention, the risk value comprises a probability of the target patient developing a post-surgical complication.

According to an aspect of the invention, there is disclosed a system for assessing a risk value for a target patient, the system comprising: a server, one or more processors, and a memory, the one or more processors communicatively coupled to a database, the database comprising historical data and time-marked data from a plurality of patients; the one or more processors configured to receive, via a connection mechanism, target patient data; and, the memory comprising instructions, that, when executed by the one or more processors, configures the server to: receive, at the server, said target patient data; pre-process or process, via a processor, said target patient data; estimate, using one or more risk assessment models, one or more risk values associated with the target patient data.

According to an embodiment of the invention, target patient data comprises one or more of: historical data, patient population-level data, and real-time data.

According to an embodiment of the invention, the one or more risk assessment models comprise one or more of: a historical data risk assessment model, a real-time data risk assessment model, and a combined risk assessment model, the combined risk assessment model comprising one or more risk assessment models.

According to an embodiment of the invention, the historical data risk assessment model is trained by: receiving, at a server comprising one or more processors and a memory, historical data corresponding to a plurality of patients, the one or more processors comprising one or more of a mapping engine and a standardization engine, the historical data for each patient of the plurality of patients corresponding to an indication of whether a patient encountered a complication; generating pre-processed historical data by: mapping, via the mapping engine, the historical data for each patient of the plurality of patients, to numerical values; standardizing, via the standardization engine, historical data for each patient of the plurality of patients; performing a regression analysis on the pre-processed data, the regression analysis determining a relationship between historical data and a risk value.

According to an embodiment of the invention, the real-time data risk assessment model is trained by: receiving, at a server comprising one or more processors and a memory, time-marked data corresponding to signal data measured by a plurality of sensors coupled to a plurality of patients, the one or more processors comprising one or more of a filtering and augmentation engine and a standardization engine, the time-marked data for each patient of the plurality of patients corresponding to an indication of whether a patient encountered a complication; generating pre-processed time-marked data by: filtering and augmenting, via the filtering and augmentation engine, the time-marked data for each patient of the plurality of patients; standardizing, via the standardization engine, the time-marked data for each patient of the plurality of patients; performing one or more regression analyses on the pre-processed time-marked data, the one or more regression analyses determining one or more relationships between real-time data and a risk value. According to an embodiment of the invention, the risk value comprises a probability of the target patient developing a post-surgical complication.

According to an embodiment of the invention, the system further comprises a display system for displaying risk value.

According to an aspect of the invention, there is disclosed a non-transitory computer-readable storage medium, the computer-readable storage medium including instructions that when executed by a computer, cause the computer to the steps of: receive, via a connection mechanism, target patient data; pre-process or process, via a processor, said target patient data; estimate, using one or more risk assessment models, one or more risk values associated with the target patient data.

According to an embodiment of the invention, the target patient data comprises one or more of: historical data, patient population-level data, and real-time data.

Other features will become apparent from the drawings in conjunction with the following description.

The advantages and features of the present invention include convenient, early management to handle complications such as anastomotic leakages, using techniques disclosed herein, that may significantly mitigate risks associated with such complications. The advantages and features will become better understood with reference to the following more detailed description and claims taken in conjunction with the accompanying drawings in which like elements are identified with like symbols.

### BRIEF DESCRIPTION OF THE DRAWINGS

Several embodiments of the present disclosure will be provided, by way of examples only, with reference to the appended drawings, wherein:

FIG. 1 is a representative ROC curve with the greatest Youden Index threshold highlighted by a black dot.

FIG. 2 shows baseline pH thresholds per hour.

FIG. 3 shows mean pH.

FIG. 4 shows the continuous learning of the method in accordance with one embodiment.

FIG. 5 shows a method to determine the post-operative complication probability after the method has been initialized.

FIG. 6 illustrates a method for assessing a risk value for a patient.

FIG. 7 illustrates a system for assessing a risk value for a patient.

FIG. 8 illustrates a method for training a historical data risk assessment model.

FIG. 9 illustrates a method for training a real-time data risk assessment model.

FIG. 10 illustrates a method for estimating a risk value of a patient.

### DETAILED DESCRIPTION

Various implementations and aspects of the specification will be described with reference to details discussed below. The following description and drawings are illustrative of the specification and are not to be construed as limiting the specification. Numerous specific details are described to provide a thorough understanding of various implementations of the present specification. However, in certain instances, well-known or conventional details are not described in order to provide a concise discussion of implementations of the present specification.

Various apparatuses and processes will be described below to provide examples of implementations of the system disclosed herein. No implementation described below limits any claimed implementation and any claimed implementations may cover processes or apparatuses that differ from those described below. The claimed implementations are not limited to apparatuses or processes having all the features of any one apparatus or process described below or to features common to multiple or all the apparatuses or processes described below. It is possible that an apparatus or process described below is not an implementation of any claimed subject matter.

Furthermore, numerous specific details are set forth in order to provide a thorough understanding of the implementations described herein. However, it will be understood by those skilled in the relevant arts that the implementations described herein may be practiced without these specific details. In other instances, well-known methods, procedures and components have not been described in detail so as not to obscure the implementations described herein.

In this specification, elements may be described as “configured to” perform one or more functions or “configured for” such functions. In general, an element that is configured to perform or configured for performing a function is enabled to perform the function, or is suitable for performing the function, or is adapted to perform the function, or is operable to perform the function, or is otherwise capable of performing the function.

It is understood that for the purpose of this specification, language of “at least one of X, Y, and Z” and “one or more of X, Y and Z” may be construed as X only, Y only, Z only, or any combination of two or more items X, Y, and Z (e.g., XYZ, XY, YZ, ZZ, and the like). Similar logic may be applied for two or more items in any occurrence of “at least one . . . ” and “one or more . . . ” language.

In some embodiments, sensors may include electrochemical or solid-state sensors with different forms, which include but are not limited to potentiometric, voltammetric, conductometric, capacitive, amperometric or ion-sensitive field effect transistors (ISFET). In some embodiments, sensors may be piezoelectric or micro-electro-mechanical systems (MEMS). Sensors may include terminals that connect to active, counter, reference or pseudo-reference electrodes depending on the type of sensor being utilized. Sensors can be of different types that include but are not limited to pH sensors, ion-sensitive sensors, temperature sensors, lactate sensors, electrolyte sensors, impedance sensors, light-based sensors, microorganism sensors, protein sensors, DNA-based sensors, carbohydrate sensors, enzyme sensors, oxygen sensors such as P02 (partial pressure of oxygen) sensors, amylase sensors, urea sensors, creatinine sensors, motion sensors (such as accelerometers), pressure sensors and flow sensors.

Sensors may be connected in series or in parallel, and may be disposed sequentially, for example, along a length of a fluid channel.

In some embodiments, sensors may include one or more temperature sensors, such as thermistors or resistance temperature detectors.

In use, temperature sensors may undergo changes in resistance correlated to changes in temperature. Thus, a temperature may be determined by determining a resistance of the thermistor, by exciting with current and measuring voltage (or vice versa).

In some embodiments, sensors may include a pH sensor that is electrochemical in nature allowing biological analytes to be transduced into electrical signals that can be then measured, monitored and analyzed to determine if a postoperative complication is developing. A system of interdigitated electrodes (active, counter and reference) may be fabricated on a biocompatible substrate. The electrodes may be fabricated from biocompatible materials: gold, platinum, titanium and silver, and then later functionalized with an active polyaniline (PANI) polyaniline/polyurethane (PAIN/PU), polyurethane, polymer or other suitable layer. In an example, m-biosensors are 500 pm×500 pm in size, allowing them to be placed on or embedded in catheters to monitor changes in pH over time.

In some embodiments, sensors may include a light-based sensor, such as photoelectric sensors, utilizing a combination of light transmitters or sources and detectors in the ultraviolet to infrared spectrum to measure the fluid's light absorption or transmission characteristics. Single-wavelength or multi-wavelength rays may be used.

In some embodiments, sensors include an impedance sensor typically operated with an alternating current (AC) excitation which may be used to evaluate the user status. An impedance sensor may include an electrode pair, and include an excitation and readout circuit.

### Assessing Post-Operative Risk

Systems, methods and devices disclosed herein can be utilized for monitoring, detecting and predicting different forms of postoperative complications, such as leakage, that can arise following surgeries. Embodiments can include a sensing and diagnostic device that utilizes sensors, for example, on a catheter or an inline device, to detect or predict, for example, the presence of luminal fluid when a leak develops.

In some embodiments, systems, methods and devices disclosed herein include sensors, such as biosensors that can be used to sense bio-signal data, placed at locations proximate to the surgical site, enabling the monitoring of biological fluids for analytes that could be indicative of a surgical leak.

Bio-signal data may be received from prior art sensing systems, for example those described in US Publication 2022/0265175, which is owned by the assignee of the present application and is incorporated herein by reference in its entirety.

In some embodiments, sensors may include electrochemical or solid-state sensors with different forms, which include but are not limited to potentiometric, voltammetric, conductometric, capacitive, amperometric or ion-sensitive field effect transistors (ISFET). In some embodiments, sensors may be piezoelectric or micro-electro-mechanical systems (MEMS). Sensors may include terminals that connect to active, counter, reference or pseudo-reference electrodes depending on the type of sensor being utilized. Sensors can be of different types that include but are not limited to pH sensors, ion-sensitive sensors, temperature sensors, lactate sensors, electrolyte sensors, impedance sensors, fluid sensors, light-based sensors, microorganism sensors, protein sensors, inflammatory sensors, carbohydrate sensors, enzyme sensors, oxygen sensors such as P02 (partial pressure of oxygen) sensors, amylase sensors, urea sensors, creatinine sensors, pressure sensors and flow sensors.

Sensors may be connected in series or in parallel, and may be disposed sequentially, for example, along a length of a fluid channel.

In some embodiments, sensors may include a temperature sensor, such as a thermistor.

In use, a thermistor may undergo changes in resistance correlated to changes in temperature. Thus, a temperature may be determined by determining a resistance of the thermistor, by exciting with current and measuring voltage (or vice versa).

A temperature sensor may be used to account for a number of artifacts and error sources in the biosignal measurements. A temperature sensor may be used to compensate or modulate signals from other sensors that are temperature dependent such as impedance and pH. A rise in fluid temperature detected by temperature sensor can indicated an influx of new fluid, as biological fluids tend to have higher temperatures relative to ambient temperatures.

An array of temperature sensors and a heating element may be used to measure fluid flow rate using the principles of thermal mass fluid transport

In some embodiments, sensors may include a flow sensor such as a flowmeter to measure the volumetric or mass flow rate of a fluid such as a liquid or a gas, for example, in a user's body.

In some embodiments, sensors may include a pH sensor that is electrochemical in nature allowing biological analytes to be transduced into electrical signals that can be then measured, monitored and analyzed to determine if a postoperative complication is developing. A system of interdigitated electrodes (active, counter and reference) may be fabricated on a biocompatible substrate. The electrodes may be fabricated from biocompatible materials: gold, platinum, titanium and silver, and then later functionalized with an active polyaniline (PANI) polyaniline/polyurethane (PAIN/PU), polyurethane, polymer or other suitable layer. In an example, m-biosensors are 500 μm×500 μm in size, allowing them to be placed on catheters to monitor changes in pH over time.

A pH sensor may be formed from a conducting polymer made from Aniline monomers. A sensitivity to pH levels of a suitable conducting polymer can allow for its use as a pH sensitive component in pH sensors.

A pH sensor may be calibrated and/or controlled by a potentiostat, in particular, an electronic device that controls the difference in potential and current of a 3-electrode system comprising of a working electrode (WE), a reference electrode (RE) as well as a counter electrode (CE). This electrical instrument has many applications that may be used to fabricate a pH sensor such as Cyclic Voltammetry (CV), Chronoamperometry and Chronopotentiometry.

A pH sensor may be configured to detect a pH value within a threshold or boundaries, or deviation from such boundaries.

In some embodiments, sensors may include a light-based sensor, such as photoelectric sensors, utilizing a combination of light transmitters or sources and detectors in the ultraviolet to infrared spectrum to measure the fluid's light absorption or transmission characteristics. Single-wavelength or multi-wavelength rays may be used.

Light-based sensors can include a combination of light transmitters and detectors in the ultraviolet to infrared spectrum and be used to measure a fluid's light absorption or transmission characteristics.

Light absorption or transmission characteristics can be indicative of changes in the bodily and luminal fluids that can include, but are not limited to, protein composition and concentration, pH, conductivity, inflammatory markers and cellular activities due to onset of complications or disease. This also enables measurement of the fluid's color, which can be indicative of bleeding (red), bile leaks (green-yellow), fecal leaks (brown), gastric leaks (green), urine leaks (yellow), and other fluids of specific colors.

In some embodiments, single-wavelength or multi-wavelength rays may be used. Changes detected in the absorption or transmission characteristics of fluids within specific light bands or wavelength may enable measurement of a fluid's color. Since serous fluids (for example, peritoneal and pleural fluids) are typically pale yellow, a change in color may be indicative of bleeding (red), bile leaks (green-yellow), fecal leaks (brown), gastric leaks (green), urine leaks (yellow), or other fluids of specific colors.

In some embodiments, a light-based sensor may include a combination of light transmitters and detectors in the ultraviolet to infrared spectrum to measure the scattering of light by the fluid to measure its turbidity. Serous fluids are typically clear in appearance and low in turbidity. An increase in turbidity, for example, as measured as an increase in the light measured by a photodetector at right angle, may be indicative of white blood cells and microorganisms within the fluid, which may be due to infection.

A light-based sensor may include multiple light sources and receivers. For instance, a single broadband light source may be used in combination with multiple band-specific photodiodes (e.g., red, green and blue). In this way, the absorption/transmission characteristics of the fluid can be measured across as many bands as there are photodetectors present. Similarly, multiple light sources may be utilized in combination with a single broadband photodetector, whereby each light source is turned on successively and the transmitted light measured accordingly by the photodetector. Lastly, light sources and photodetectors may also utilize dynamic filters to allow the emission or detection of specific bands of light in lieu of multiple sources or photodetectors.

In some embodiments, sensors include an impedance sensor typically operated with an alternating current (AC) excitation which may be used to evaluate the user status. An impedance sensor may include an electrode pair, and include an excitation and readout circuit.

In some embodiments, an impedance sensor may be configured to perform AC excitation within a well-defined and constant fluid geometry (constrained by a channel or housing), allowing a normalized impedance (or specific impedance) and admittance to be determined.

In some embodiments, a fluid's impedance may be measured across a range of frequencies (ranging from Hz to MHz) to separate the contribution of individual electrolytes and infer the ionic composition of the fluid. A user condition may be based at least in part on the fluid's ionic composition.

Measured impedance values may be transformed to determine a conductivity (for example, real element of the impedance) of a fluid. Conductivity may reveal a characteristic of the fluid itself, and hence may directly serve a clinical value.

For example, conductivity may indicate an analyte's inherent characteristics and composition.

Impedance may be affected by fluid volume and geometry, and thus measured impedance may be used to localize and track particles and bubbles in a fluid channel.

In some embodiments, an impedance sensor may be used to account for a number of artifacts and error sources in bio-signal measurements.

In some embodiments, an impedance sensor may be used to detect a rapid and drastic increase in impedance beyond the range of bodily fluids which may be indicative of the presence of air bubbles in the channel. Air bubbles are a challenge to catheter-based measurements as they cause artifacts with readings.

In some embodiments, an impedance sensor may be used to detect a sudden increase in impedance, which may be indicative of a presence and a quantity of non-homogenous substances and particles (e.g., blood clots, fibrin).

In some embodiments, an impedance sensor may be used to detect blood coagulation (typically characterized by a sudden increase in impedance, followed by a slower but sustained increase in impedance), and hence, the presence of blood and risk of channel blockage.

In some embodiments, an array of impedance sensors placed along the channel may be used to detect and track air bubbles, non-homogenous substances, and/or particles as they travel through the channel, using techniques described herein.

In some embodiments, sensors may include amylase sensors.

In use, systems, methods and devices may monitor for trends and changes in physical and chemical biomarkers that may include but are not limited to pH, temperature, fluid flow, pressure, lactate, lactic acid, nitrates, glucose, alkali ions, oxygen, bicarbonate, inflammatory proteins, bacterial proteins and other biomarkers, for example, that are associated or correlated with leakage.

Single sensors or sensor arrays can be placed along the wall of a catheter, inside dedicated lumens, or in an inline device, that enable the device to detect and monitor if a leak is developing.

In some embodiments, a catheter may be used as a carrier for sensors to monitor the internal compartments of the body such as the peritoneal or pleural cavity, without applying any negative pressure. Catheter may be connected to a balloon or a mechanical pump to apply negative pressure to facilitate the drainage of fluid. A catheter may also be connected to a fluid supply such as saline solution to perform therapeutic and diagnostic functions such as dialysis or irrigation.

In some embodiments, multiple sensors may be spaced apart along a length of a catheter. Multiple sensors placed along the catheter, may allow for multiple regions to be sensed and spatial progression of a leak to be tracked.

A catheter may be formed of a tube having a hollow or solid body and made of medical grade materials, such as a suitable polymer. In some embodiments, the catheter may be a flexible substrate.

In some embodiments, a catheter may be formed of a material with low friction.

A catheter may have different designs where the catheter may be cylindrical, rectangular, flat, or T-shaped in cross-section and the catheter may have a single lumen or multiple lumens.

In some embodiments, sensors may be disposed inside reservoirs where fluids may be collected from a user's body. Reservoirs can include elements such as balloons, pumps or other containers that may hold biological fluids. Sensors disposed within a reservoir can be used simultaneously with sensors placed in catheters. This may allow for more sensors to be utilized to determine a variety of different conditions or post operative complications such as fluid leakage, infection, inflammation or other dangerous complications.

Sensors such as biosensors may be connected to a monitor such as an electronic data acquisition system (DAQ) that may be situated inside or outside a user's body, which may continuously process data obtained from the sensors. The connection can be established via different methods including but not limited to, wires and connectors that may be embedded within the catheter's body or within at least one lumen designed to allow wires and connectors run through them. The connection may also be established wirelessly by transmitting the data obtained in-vivo from biosensors via a transmitting system to a receiver placed outside the body.

In some embodiments, each of multiple sensors are independently in communication with a monitor.

A monitor may have a screen allowing readouts to be directly observed on the device. A monitor may also use various visual or audio queues such as small LEDs or alarm sounds to signal various events.

Data acquired by a monitor can also be communicated to a computer system via wired or wireless media to allow further analysis and visualization. The data communicated may be processed, raw, or summarized.

In some embodiments, data collected by a monitor can be analyzed to identify trends associated with the development of different complications. This may be performed by evaluating single or multiple data sets acquired from one or more sensors over time to diagnose and determine the stage of development of the complications.

Should one or more of the sensors demonstrate biological trends that are associated with surgical leakage, an alarm signal may be sent from the monitor to a computer-based system allowing users to determine the appropriate medical action.

In an example, a slow decrease in local pH could indicate either a small leak or poor blood supply to the wound site. If a simultaneous slow increase in lactate concentration is observed, it may indicate a lack of blood supply (i.e., ischemia). If lactate concentration is steady, it may indicate a slow leak.

In another example, a sharp decrease in pH may indicate a large leak. If the pH returns to its baseline, it may suggest that a wound is healing despite the leak. If the pH continues to drop, or remains low, it may indicate a significant leak that the body may have difficulty recovering from.

Systems and methods disclosed herein may perform monitoring, detection and diagnosis, and prediction. For example, monitoring may present data that is sensed by sensors such as biosensors. Detection and diagnosis may, by way of algorithms, detect a condition in a user and/or make a determination of a diagnosis, such as a leak, what kind of leak it is, and where the leak came from, for example, with an associated confidence level. A prediction may use sensory data to examine different trends and process signals to predict a leak that may occur in the future, for example, with an associated confidence level. As such, embodiments of systems and methods disclosed herein may identify physiological differences between a leak occurring and precursors to a leak.

Systems and methods disclosed herein may be used to perform clinical functions. In an example, a catheter system may be connected to mechanical elements that can apply negative pressure allowing fluid to be drained from a user's body in addition to its diagnostic function. Such clinical function can be both performed at locations in a user body such as inside a GI tract or in a peritoneal cavity.

Techniques for applying negative pressure may include but are not limited to balloons, mechanical pumps, vacuum systems or other devices that can suck fluid, for example, from the body to the outside. In some embodiments, fluid that is being drained may assist in diagnostic application by causing constant fluid flow across sensors. In some embodiments, a clinical function may be performed by pumping fluid into a user's body.

The term “bodily fluid(s)” as used herein may refer to fluids originating from inside the human body, fluids that are excreted or secreted by a body (e.g., blood, gastric juice, and peritoneal fluid), and similar fluids. In extension, the term “luminal fluid” refers to a subset of bodily fluids that exist within inner cavities, intestines, vessels, tubular organs and many other membrane-bound organs such as gastric juices, intestinal fluids, fecal matter, urine, bile fluid, and other similar fluids.

The terms “biomarker(s)” and “aptamer(s)” as used herein may refer to molecules, substances, and chemical or physical properties that can be measured or detected as bio-signals in bodily fluids. They include, but are not limited to, pH, temperature, electrolyte concentration, fluid flow rate, pressure, lactate, lactic acid, nitrates, alkali ions, inflammatory proteins, bacterial proteins, specific cells, molecules, genes, gene products, enzymes, hormones, inflammatory proteins, and glucose.

The terms “biosensor(s)” and “sensor(s)” as used herein may refer to a device or system that detect or react to biomarkers or bio-signals, transducing these signals into measurable electrical signals. Biosensors and sensors utilized herein may include but are not limited to pH sensors, lactate sensors, amylase sensors, lactic acid sensors, glucose sensors, temperature sensors, pressure sensors, enzymatic sensors, protein sensors, biological sensors, ion sensors, electrolyte sensors, impedance sensors, conductivity sensors, flow sensors and other forms of electrochemical and solid-state sensors.

In some embodiments, signal data received from a sensor device may be aggregated and associated with a user, for example, over time, to develop a profile for that user. In some embodiments, user profile information, such as signal data associated with one or more users, may be applied to machine learning techniques to develop models for such signal data.

A user profile may include information about a user such as information related to a surgical procedure performed on the user and date and time of the surgical procedure, location of the surgical procedure, date and time of insertion of the sensor device, location of insertion of the sensor device, the user's age, height, weight, medical history, condition or illness (e.g., diabetic), current or past medication in use by the user, or other current or historical factors related to a user, surgery, or device details.

In some embodiments, a user profile includes a list of medications used by the user. This may be used to identify potential error sources caused by medication altering the threshold of one or more of the bio-signals being measured using sensors described herein.

In some embodiments, a user profile comprises the procedures that were performed on the patient. Such a list of procedures can be used to further analyze the potential list of complications that the user may suffer from given the risks for each procedure. Furthermore, such a list of procedures may be used to identify the anatomy of biological fluids proximate to the procedure location.

In some embodiments, information related to a surgical procedure performed on the user includes a date and time of the surgical procedure. Furthermore, the surgery date and time may be used to analyze the user condition given the full timeline of recovery for the user.

In some embodiments, a user profile data may be input by the user, a healthcare institution, or may be input by a healthcare professional, for example, a surgeon may input information related to a surgery that was performed and details regarding the sensor device (for example, operating parameters, the number and type of sensors, etc.) being used following a surgery.

In some embodiments, a user profile may be automatically generated, for example, from health records indicating surgical details, or a user's electronic health record. These may be received from a computing device in communication with system 100.

In some embodiments, a user profile may include information identifying factors that are associated with a user condition determined from collected signal data.

In an example, a user condition dictated by sensor data that occurs for a temporary period of time, indicating, for example, a temporary spike, may be discarded as not indicating that particular condition, and instead an anomaly.

In some embodiments, the future occurrence of a complication, such as an anastomotic leak, can be predicted based at least in part on a time at which the user condition occurs, and a length of time for which the user condition occurs.

Machine learning algorithms may be applied to previously acquired signal data associated with a user condition. For example, pattern recognition may be performed on previously acquired signal data that is associated with a particular user condition. The machine leaning may generate a user condition classification model trained by the previously acquired signal data.

Further description of such models will be described in greater detail below.

A leakage may be predicted by an analysis of a change in flow of fluid surrounding a sensor of the sensor device. For example, how fast a change in flow occurs may be indicative of how fast a leak is flowing.

In another example, a leak may be predicted on the basis of a build-up of lactate detected by a sensor.

In another example, a leak may be predicted on the basis of a depletion of oxygen detected by a sensor.

In another example, a leak may be predicted on the basis of a detected pH change, and may include an analysis of the why the pH has changed to differentiate between different causes or conditions for such a pH change.

In some embodiments, a future occurrence of the anastomotic leak may be predicted based on a user's condition being above or below a predetermined threshold. Such a threshold may be, for example, a pH value.

As described above, a secondary condition or second user condition may be determined from sensor data. The future occurrence of a leak may be predicted based at least in part on the second user condition. The second user condition may also indicate a risk factor or risk level of a leak condition.

In some embodiments, risk models may determine confidence levels for whether a leak has occurred or not, based on a combined analysis of the user profile and processed biosignals. Weighted coefficients based on the user's profile and current condition may be used to contextualize algorithm inputs/outputs depending on the likelihood that a leak is developing. These weights may be dynamic over time and updated as the user's condition is updated. In an example, if a user had undergone bariatric surgery, higher weights may be applied to gastric leak detection algorithms, as compared to colorectal leak detection algorithms.

In some embodiments, predictive analysis of signals such as biosignals from a biosignal sensor, such as from a sensor device or inline monitoring device, may include a diagnosis, for example, an identification of the nature of a leak or illness by examination of symptoms monitored by a sensor.

In some embodiments, a triage condition or risk level of a future occurrence prediction of a leak may be based on the signal data, the user condition, and the user profile. The data generated may include a triage condition or a risk level.

Machine learning algorithms may be applied to previously acquired signal data, user profile data, and user condition data. For example, pattern recognition may be performed on previously acquired signal data that is associated with a particular leak prediction.

Data associated with a future occurrence prediction of a leak may include a notification of the prediction.

In some embodiments, signal data may be collected to build a trend across a number of patients, and a cross-correlation technique may be used to identify the similarity between a patient's data and previous patients. And a match or correlation may indicate a risk factor.

Conveniently, it may be possible to make better predictions because there is a better fidelity of data acquired over time, for example, continuously as fluid flows through a sensor device, and in an example, in real-time or near real-time.

An example application for systems and methods disclosed herein can be showcased by looking at a patient that is suffering from colorectal cancer, and the tumor needs to be removed. The surgeon may decide to perform an anastomosis after removing the tumor from the body. The surgeon may then place the catheter with biosensors in one of the paracolic gutters if they think it is the most likely region to collect fluid from a leak. The catheter system that they utilize may be equipped with fluid, pH and lactate sensors. Once the catheter has been placed, the surgeon may also choose to use absorbable sutures to keep the catheter held in place. The catheter may then be connected to a monitor placed outside the body. The catheter may also connect to a balloon which would apply negative pressure to drain fluid from the peritoneum.

The monitor would then confirm that a connection has been established with the biosensors, informing the user and the surgeon that the patient condition appears to be normal. The patient may then be kept in the hospital overnight and then discharged the second day. The patient may be discharged with the monitor and the catheter. Three days following the surgery and after the patient has been discharged the biosensors may detect clinically relevant pH change and simultaneous increases in lactate concentration. The monitor may then signal to the patient to seek medical attention, or the system may delay the signal to wait for more significant changes. The data may be relayed wirelessly to the medical facility, at which point a specialist may look at the data and also make a decision of whether to have the patient come to the medical facility or not. The system may then detect a significant pH change and a relatively high flow of fluid in the abdominal cavity. The monitor may then alert the patient informing them that a leak has been detected and that they need to seek medical intervention immediately. The medical facility may also be notified. Once the patient is at the hospital, the medical facility may look at the data obtained from the biosensors and make a clinical decision to support the patient before the complication grows. A surgeon may also decide to take corrective medical action, including but not limited to re-operation on the patient. The system may be utilized again after the corrective action is done.

Conveniently, a sensor device as described herein may be removable from a user or patient in an outpatient setting, for example, in a user's home by a nurse, and without the need for a user to undergo an additional surgical procedure. In an example, this may occur ten to twenty days following a surgery.

In some embodiments, integrating sensors with catheters (and specifically, drains) may allow a sensor device to be removed in an outpatient setting.

The methods calculate continuous, real-time numeric score based on pH of drainage fluid that is related to the probability of patients developing post-operative complications such as leaks.

Sets of pH measurements are obtained from patients (i.e., from patient biofluids) in a clinical trial, either continuously or at discrete time intervals. Each patient's data will be labelled with “Leak” (AL) or “non Leak” (nAL). Continuous data may be subdivided into time intervals of any length. For this example, the intervals were 1-hour intervals. The determination of baseline threshold values is done using Receiver Operator Characteristic (ROC) analysis based on pH per post-operative hour from the clinical data.

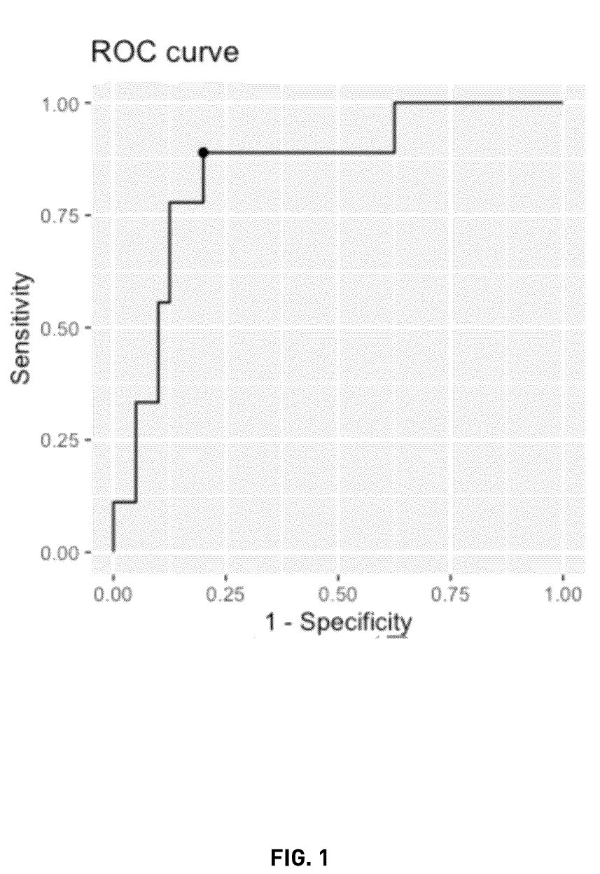

FIG. 1 illustrates an ROC analysis of the data. Essentially, data from each hour is examined and the baseline pH value for that hour interval is determined, via ROC analysis, thereby allowing to differentiate between AL and nAL patient data. The baseline is based on the greatest area under curve (AUC) of the ROC curve or based on the highest possible sum of sensitivity and specificity. A representative ROC curve with the greatest Youden Index threshold is highlighted by black dot.

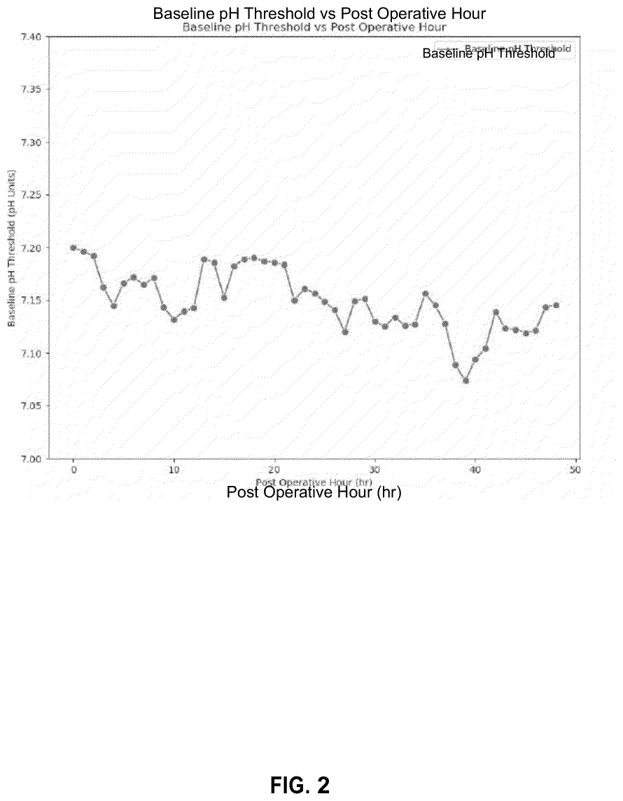

FIG. 2 illustrates baseline cut off points per interval. This step is repeated until all available data has been processed. The result would be a list of baseline cut off points per interval (graphically shown on FIG. 2, showing baseline pH thresholds per hour). Each point represents a pH value threshold that, when applied to the data in that post-operative hour interval, will discriminate between patients with nAL and AL, for example, by maximizing the sum of the sensitivity and specificity of the data, or other methods which may estimate a maximum true positive rate with minimum false positive rate.

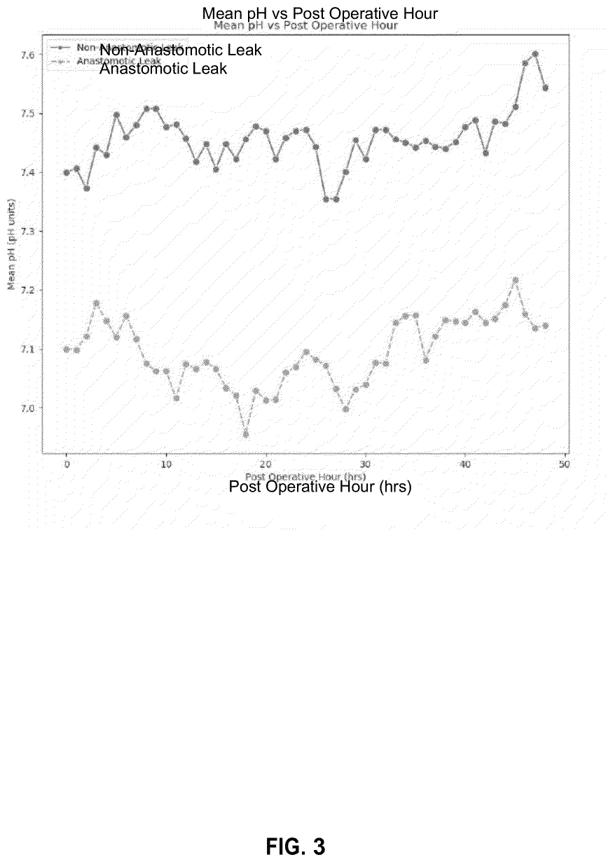

In FIG. 3, mean pH is illustrated as a function of post-operative hour.

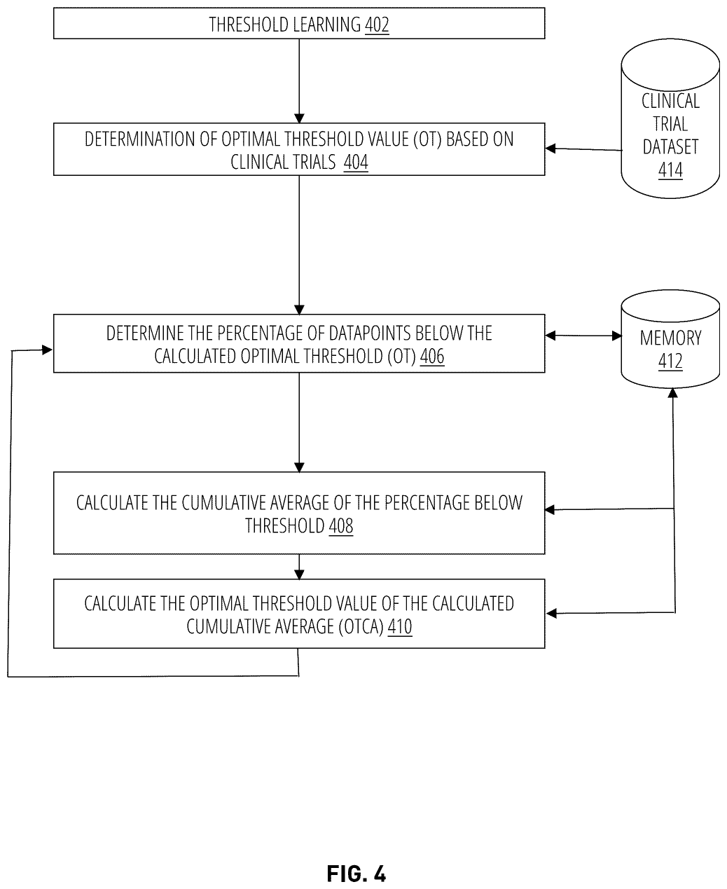

FIG. 4 illustrates an embodiment of a method to calculate continuous, real-time numeric score based on pH of drainage fluid that is related to the probability of patients developing post-operative complications, is described.

The method allows healthcare providers (HCP) to get a real-time status of their patients for closer monitoring, intervention or discharge earlier compared to the SOC.

The method comprises continuous learning, depicted in the flow chart of FIG. 4. The method learns from datasets of clinical trials and ongoing usage as per FIG. 2. The baseline threshold (OT) and baseline threshold cumulative average (OTCA) is initially set up based on a dataset 414 from clinical trials based on biomarkers statistics of patients that had complications (e.g., leak) versus those who did not have complications.

The method learns on an ongoing basis based on data gathered in the memory 412 from usage of the embodiment by patients (see FIG. 2) and whether or not they encountered complications.

The method measures one or more biomarkers (pH is used as an example to describe the embodiment, but the method is not limited to pH) data points at regular intervals (for example each 5 minutes). The data points are stored in memory 412.

The method calculates a baseline threshold (OT) value for the pH data points using Receiver Operator Characteristic (ROC) analysis 404.

The method determines the percentage of data points 406 that fall below the calculated baseline threshold in each interval. A cumulative average (CA) of the percentage below the calculated baseline threshold 408 is calculated for each interval.

The baseline threshold value of the calculated cumulative average (OTCA) is calculated using Receiver Operator Characteristic (ROC) analysis 410.

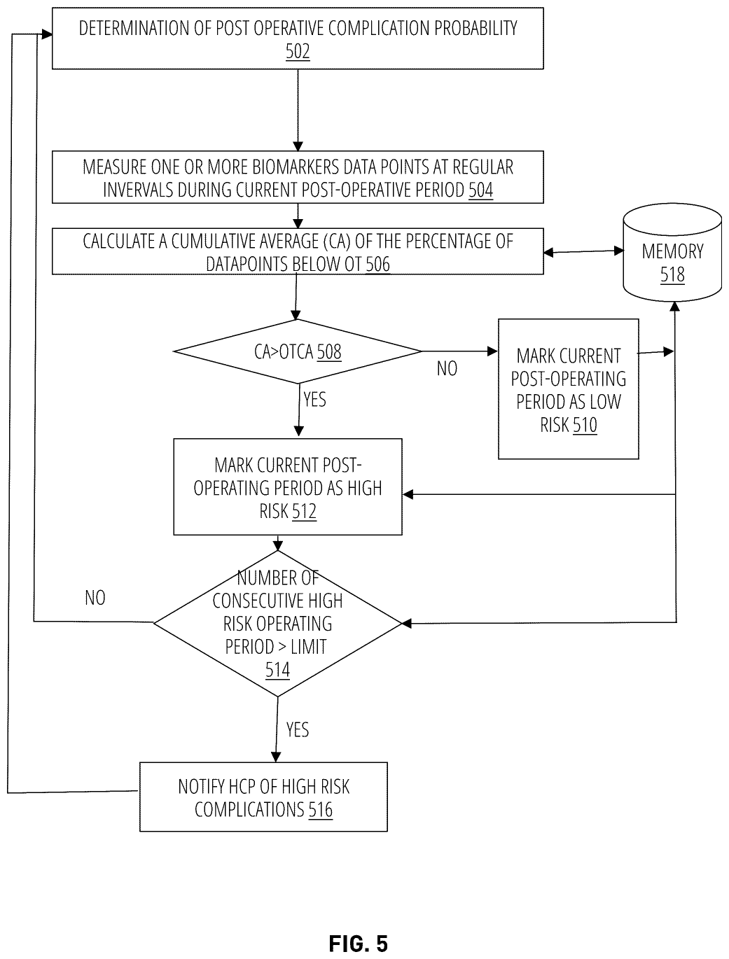

FIG. 5 depicts a method to determine the post-operative complication probability 502, after the method has been initialized.

The method measures one or more biomarkers at regular intervals (for example each 5 minutes) during a current post-operative period.

The method calculates the percentage of data points in the current post-operative period interval that fall below OT and calculates the CA of the percentage of data points below OT 506.

The current post-operative hour interval is identified as “High Risk” 512 if CA exceeds OTCA 508. Otherwise, mark as “Low Risk” 510.

If the number of consecutive post-operative hours marked as “High Risk” exceeds a predetermined limit (e.g., 10), the method notifies HCP that patient has a high risk of having or developing a post-operative complication by sending a message, effecting a noise on a device etc. 516.

If a patient develops complications, the dataset accumulated for this patent is marked as such, in order to improve the accuracy of the CA and OTCA thresholds.

The post operative period can be 30 minutes, one hour, half a day etc.

IIR (infinite impulse response) or FIR (finite impulse response) temporal filter can be used instead of a CA to calculate OTCA. For example, such as moving average, gaussian window, or exponential filter.

Other non-linear statistical methods such as median or mode can be used to determine OT.

Other pre-, intra- and post-operative data (e.g., American Society of Anesthesiologists (ASA) score, demographics, surgery details, etc.) can also be integrated in the method.

The method can combine more than one biomarker data or other sources data that is available to the HCP (e.g., EMR/EHR, heart rate, other sensors or medical devices) to give a more accurate probability score. Different weights can be applied to different biomarkers to tune their impact on output risk/prediction. Other meta-data about the patient can be integrated into method. Statistical confidence can be added to measured probability.

The method can be deployed in firmware, executed on a processor or on a cloud server allowing updates and improvements. The method can be adapted to detect different types of post operative complications. The method can be used with pH data from any source.

### Developing Models for Assessing Post-Operative Risk

Embodiments disclosed can include a computer implemented method for predicting a post-operative risk value for a patient, based on analyzing continuous patient data and the patient's historical data against separate risk models comprising a plurality of patients' real-time data and risk-values, and a plurality of patients' historical data and risk-values.

In some embodiments, computer implemented methods may be used in conjunction with a target patient's pre-existing medical records, electronic medical records (EMRs), pre-surgical patient data, and the like.

As in the previous section, bio-signal data may be received from prior art sensing systems, for example those described in US Publication 2022/0265175, which is owned by the assignee of the present application and is incorporated herein by reference in its entirety.

Generally, for the purposes of this disclosure, a target patient is a patient who is being monitored for complications and/or risk associated with conditions pre, post, or during surgery.

Additionally, a plurality of patients may provide time-marked data and historical data in order to train various risk models disclosed herein. This data may be obtained from clinical trials, medical records, and the like.

In some embodiments, computer implemented methods may be used in conjunction with inline devices, which may continuously measure, with sensors, real-time data of a target patient or of a plurality of patients, the real-time data being sent, via a network, to a server comprising one or more risk assessment models, either to provide a risk value for the target patient, or to train the one or more risk assessment models to assess risk based on the real-time data.

Inline devices may comprise devices which are inline with a catheter, or embedded within a catheter, or placed along the catheter.

In some embodiments, computer implemented methods may be used in conjunction with implanted medical devices, benchtop devices, point of care (POC), and handheld devices.

In some embodiments, sensors may include electrochemical or solid-state sensors with different forms, which include but are not limited to potentiometric, voltammetric, conductometric, capacitive, amperometric or ion-sensitive field effect transistors (ISFET). In some embodiments, sensors may be piezoelectric or micro-electro-mechanical systems (MEMS). Sensors may include terminals that connect to active, counter, reference or pseudo-reference electrodes depending on the type of sensor being utilized. Sensors can be of different types that include but are not limited to pH sensors, ion-sensitive sensors, temperature sensors, lactate sensors, electrolyte sensors, impedance sensors, light-based sensors, microorganism sensors, protein sensors, DNA-based sensors, carbohydrate sensors, enzyme sensors, oxygen sensors such as P02 (partial pressure of oxygen) sensors, amylase sensors, urea sensors, creatinine sensors, motion sensors (such as accelerometers), pressure sensors and flow sensors.

Sensors may be connected in series or in parallel, and may be disposed sequentially, for example, along a length of a fluid channel.

In some embodiments, sensors may include one or more temperature sensors, such as thermistors or resistance temperature detectors.

In use, temperature sensors may undergo changes in resistance correlated to changes in temperature. Thus, a temperature may be determined by determining a resistance of the thermistor, by exciting with current and measuring voltage (or vice versa).

In some embodiments, sensors may include a pH sensor that is electrochemical in nature allowing biological analytes to be transduced into electrical signals that can be then measured, monitored and analyzed to determine if a postoperative complication is developing. A system of interdigitated electrodes (active, counter and reference) may be fabricated on a biocompatible substrate. The electrodes may be fabricated from biocompatible materials: gold, platinum, titanium and silver, and then later functionalized with an active polyaniline (PANI) polyaniline/polyurethane (PAIN/PU), polyurethane, polymer or other suitable layer. In an example, m-biosensors are 500 pm×500 pm in size, allowing them to be placed on or embedded in catheters to monitor changes in pH over time.

In some embodiments, sensors may include a light-based sensor, such as photoelectric sensors, utilizing a combination of light transmitters or sources and detectors in the ultraviolet to infrared spectrum to measure the fluid's light absorption or transmission characteristics. Single-wavelength or multi-wavelength rays may be used.

In some embodiments, sensors include an impedance sensor typically operated with an alternating current (AC) excitation which may be used to evaluate the user status. An impedance sensor may include an electrode pair, and include an excitation and readout circuit.

Advantageously, the system and method described below, in accordance with different embodiments, acts as an agnostic interface that allows data generated by different types of sources to be used. Thus, the system and method can be configured to work with existing systems of measuring patient biosignals.

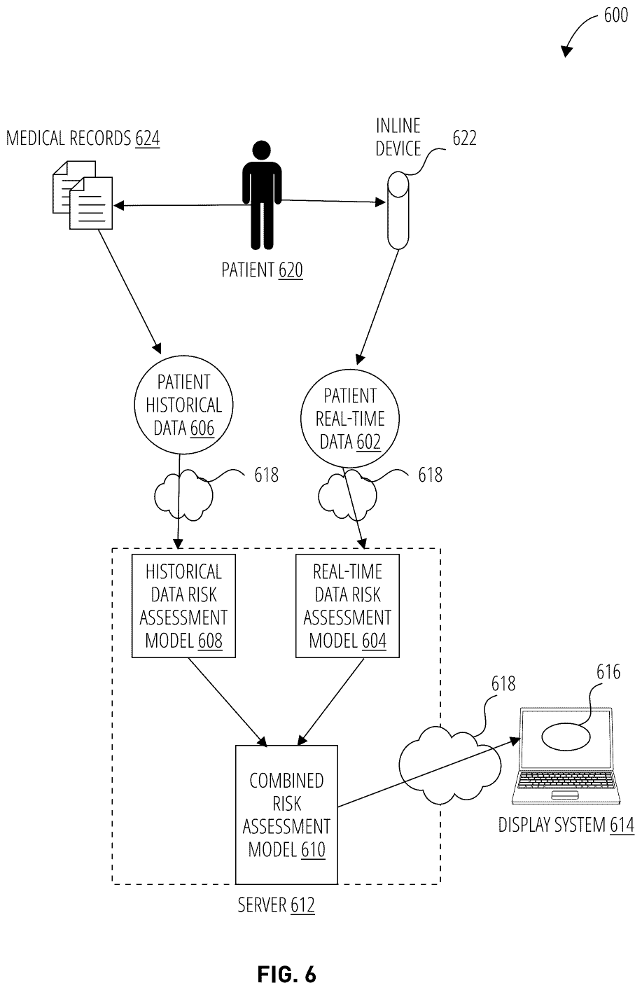

FIG. 6 illustrates a computer-implemented method for assessing a risk value for a patient 600.

Generally, a computer-implemented method 600, for assessing a risk value of a target patient 620 comprises first, receiving at a server 612, via a connection mechanism, such as a network 618, one or more of: patient real-time data 602 and patient historical data 606 from the target patient 620. The method further comprises estimating, at the server 612, a risk value 616 of a measured signal of said target patient, via a combined risk assessment model 610 comprising one or more real-time data risk assessment models 604 and one or more historical data risk assessment models 608. The method further comprises displaying, via a display system 614, or notifying a user of, the risk value 616.

The display system may comprise a computer, smart phone, physical print-out, and the like, as well as any other means of displaying data known in the art. The display system may be connected to the server via a network, a cable, or similar.

Notifying a user may comprise a notification sound, a visual notification, or similar, whether through a haptic, visual, auditory, or digital notification system.

Alternatively, the risk value may be saved without displaying or notifying a user of the risk value 616.

Risk assessment models 604 and 608 may preferably be trained separately.

Training the risk assessment models 604, 608, generally comprises sending data to a server, associating known or measured data with numerical values or indications of whether complications occurred (for example by calibrating, mapping, etc.), standardizing the data (for example, by normalizing, or subtracting a mean and dividing by a standard deviation), and performing one or more regression analyses on the standardized data, to give a model which associates a data point with a risk factor.

For example, real-time data risk assessment model 604 may be trained using continuous sensor data from a plurality of sensors measuring biosignals from a first plurality of patients. Continuous sensor data, may be sent, for example, from an inline device 622, which may continuously monitor a patient 620, sending patient real-time data 602 to the server 612 via a connection mechanism, such as a network 618, a cable, a USB device, or similar.

Alternatively, continuous sensor data may be sent from implanted devices, benchtop devices, POC devices, handheld devices, and/or medical wearables (i.e., smart watches).

Training data may comprise associating sensor data with a post-operative time, such that time-marked data may be associated with an indication of whether or not a patient from the plurality of patients suffered a complication, such that a regression curve may be developed associating sensor data with a probability of suffering a complication at a given post-operative time.

For example, time-marked data used for training the real-time risk assessment model 604 may comprise pH vs. post-operative time for each of the plurality of patients. The time-marked data may be categorized based on whether it corresponds with a patient who encountered a complication, for example. The time-marked data may be categorized based on when a given patient encountered a complication, for example. The time-marked data may be categorized by a combination of the above methods, as well as any other methods known in the art.

The historical data risk assessment model 608 may be trained using historical data, including pre-operative risk factors, from a second plurality of patients. The second plurality of patients may overlap with patients from the first plurality of patients.

The historical data risk assessment model may additionally or alternatively be trained with patient or population-level historical data.

Historical data may comprise, but is not limited to, one or more of the target patient's or target patient population's: pre-operative risk factors (e.g., demographics, weight, sex, age, etc.), medical records, surgical history, and the like.

For example, the historical data risk assessment model 608 may be trained with a plurality of patients' pre-operative data, such as their ASA scores.

Training the combined risk assessment model 610 may comprise mapping decision rules associated with the two individual models 604, 608, to risk values 616. It may further comprise decision making rules based on the availability of data within the two individual models 604, 608.

For example, the combined risk assessment model 610 may comprise a decision making engine which chooses whether to use one or both of the risk assessment models 604, 608 before calculating a risk value 616. Some patients may not have continuous, patient real-time data 602, in which case only patient historical data 606, including pre-operative data or medical records 624 may be inputted into the method 600. In this case, the decision making engine may choose to assess risk based only on the historical data risk assessment model 608.

This enables partially complete patient data to be utilized in both risk assessment model training and validation (for example, when information about a patient's pre-operative risk factors is available but not their sensor data, and vice-versa).

This is alternative to more common methods such as imputing missing values or dropping patients with missing values.

Dropping patients with missing values limits the available number of training patients, and imputing can be problematic if the number of imputed values is large compared to the number of available values and can be non-trivial for full collections of sensor measurements.

Further, the models 604, 608, and 610 may be continuously updated by training it with target patient data.

Generally, but not necessarily, the risk assessment models 604, 608, and 610, are trained using machine learning algorithms or techniques.

These may include, for example, deep learning architectures such as Deep Belief Network (DBN), Stacked Auto Encoder (SAE), Convolutional Neural Network (CNN) or Recurrent Neural Network (RNN) may be used. Other examples include, without limitation, Restricted Boltzmann machines (RBM), Social Restricted Boltzmann Machines (SRBM), Fuzzy Restricted Boltzmann Machines (FRBM), TTRBM models of Deep Belief Networks (DBN) or similar approaches could be used; AE, FAE, GAE, DAE, BAE models of Statistically Adjusted End Use (SAE) models could be used; models such as AlexNet, ResNet, Inception, VGG16, ECNN models of CNN may be used; Bidirectional Recurrent Neural Networks (BiRNN), Long Short-Term Memory (LSTM) networks, Gate Recurrent Unit (GRU) of RNN may also be used. Additional techniques specific to time-series modelling may be employed, including, but not limited to, dynamic time warping, change point detection, Autoregressive Integrated Moving Average (ARIMA).

In some embodiments, other types of algorithms such as physics-based mathematical computations and basic multiple linear regression models may also be relied upon in conjunction with or in complementarity with those architectures and learning algorithms. This may further include cumulative average (CA) methods as discussed above, and exemplified in FIG. 4 and FIG. 5.

In one, non-limiting example, the method 600 may estimate a risk value 616 for a post-surgical anastomotic leak.

The combined risk assessment model 610 uses the real-time data risk assessment model 604 and the historical data risk assessment model 608 to estimate a continuous, real-time risk value 616 of a patient to develop an anastomotic leak, generally after surgical anastomosis. In this embodiment, the historical data risk assessment model 608 comprises a “Pre-Operative Risk Factor Model”, while the real-time data risk assessment model 604 comprises a pH model. This may preferably allow users, including healthcare providers to get a real-time status of their patients for closer monitoring, intervention, or discharge earlier compared to the current standard of care.

For a target patient 620, their historical data 702, which comprises in this case pre-operative risk factors such as an ASA score of 1, 2, 3, 4, or 5, and surgical approach—for example, laparoscopy and robotic, laparotomy, is sent to a server 612 via a network 618, and is pre-processed. Pre-processing may comprise determining a pre-operative risk factor (such as ASA score) based on patient medical records. Pre-processing may comprise mapping or assigning the pre-operative risk factors to numerical values. Historical data may also comprise patients' individual health indicators, such as gender, height, weight, or past surgeries, past surgical complications, surgical parameters (including, but not limited to, duration, blood loss, etc.) and vitals data (heart rate, respiratory rate, oxygen saturation, etc.).

The pre-processed historical data may then be entered into the historical data risk assessment model 608, using standardization values calculated on data used to train the model 608. The historical data risk assessment model 608 then preferably estimates a risk value 616, based on the pre-operative risk factors, to produce a probability estimate of Anastomotic Leak Risk, using a regression model which associates a probability of leak with a given pre-operative risk factor.

For the same patient, their patient real-time data 602 may be preprocessed for a first time point or a first time period, the real-time data being, in this case, pH sensor measurements obtained from a pH sensor device which may be in fluid communication with the patient 620. For example, the pH sensor device may be continuously measuring the pH of a patient's bodily fluids. Pre-processing the pH sensor measurements may comprise taking a mean of the pH measurements over a time period. This may be achieved by filtering and augmenting the raw data with a filtering and augmentation engine.

The pre-processed patient real-time data 602 may then be entered into the real-time data risk assessment model 604, which, in this case is trained using pH sensor data.

The real-time data risk assessment model 604 may estimate a risk value, based on the pH value at the first time point or a first time period, to produce a probability estimate of Anastomotic Leak risk, using the calculated mean pHs through one or more trained pH models to produce a probability estimate of Anastomotic Leak Risk at a given time, using one or more regression models which associate a probability of leak with a given pH value for a post-operative time/time period.

The risk value may alternatively or additionally, be estimated from the CA method of FIGS. 4-5.

For the same patient, at desired time intervals (for example, at each given post-operative hour), the combined risk assessment model 610 may preferably use both models risk values to classify the patient's Anastomotic Leak Risk based on a set of rules corresponding with the two risk models.

In one, non-limiting example, the patient's Anastomotic Leak Risk may be based on the following decision rules: “Very High” if probability estimates for the Pre-Operative Risk Factor Model is above 0.5, and for the pH Model is above 0.75; “High” if probability estimates for the Pre-Operative Risk Factor Model is above 0.5, and for the pH Model is between 0.5 and 0.75; “Low” if either 1) probability estimates for the Pre-Operative Risk Factor Model is above 0.5, and for the pH Model is below 0.5 or 2) probability estimates for the Pre-Operative Risk Factor Model is below 0.5, and for the pH Model is above 0.5; “Very Low” if probability estimates for the Pre-Operative Risk Factor Model is below 0.5, and for the pH Model is below 0.5.

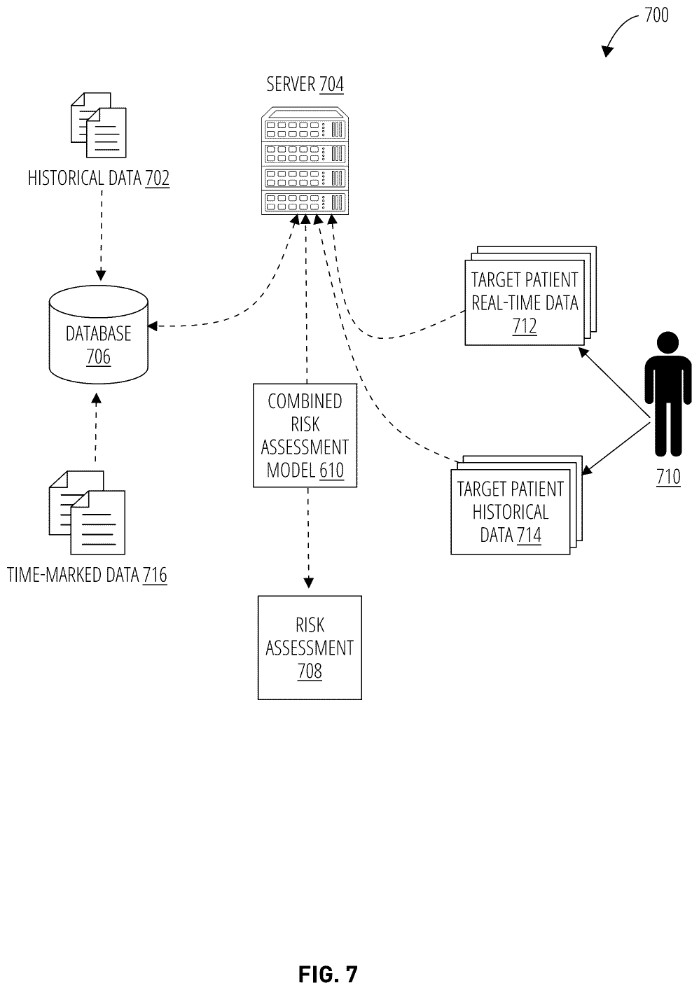

FIG. 7 illustrates a system for assessing a risk value for a patient 700.

Generally, the system comprises a server having one or more processors and a memory, wherein the one or more processors are communicatively coupled, whether via a network, a c able, or similar, to a database 706, the database comprising historical data 702 from a plurality of patients, and time-marked data 716 from a plurality of sensors measuring biosignals from a plurality of patients. The one or more processors are further communicatively coupled, via one or more networks, cables, or similar, to the target patient real-time data 712 and the target patient historical data 714.

The memory comprises instructions that, when executed by the one or more processors, configures the server 704 to:
1) Receive, at the server 704, one or more of: target patient real-time data 712 and target patient historical data 714. In terms of the target patient real-time data 712, the server may be configured to continuously receive data 712, in real-time. Alternatively, the server may be configured to continuously receive 712 at specific time intervals pre, post, or during surgery.
2) Pre-process or process, via a processor, one or more of the data 712 and 714.
3) Estimate, using a first model, a first risk value 616 associated with the target patient 710. For example, the first model may comprise the historical data risk assessment model 608, which may be trained using historical data 702 from the database 706. The historical data risk assessment model 608 may give a risk value 616 based on pre-operative risk factors.
4) Estimate, using a second model, a second risk value associated with the target patient 710. For example, the second model may comprise the real-time data risk assessment model 604, which may be trained using time-marked data 716 from the database 706. The real-time data risk assessment model 604 may give a risk value 616 based on real-time sensor measurements corresponding with the target patient's 710 physiological conditions at a given time. The second model may continuously estimate a risk value 616, for example at specific time intervals post-surgery.
5) Estimate, using a combined risk assessment model 610, a final risk value 616 associated with target patient 710, and therefore determine, based on the final risk value 616, a risk assessment 708. The risk assessment 708 may be determined based on a set of rules corresponding with the values 616 estimated in steps 3 and 4. The risk assessment 708 be determined once, continuously in real-time, or continuously at specific time intervals.

The system may further comprise a display system 614, such as a smart phone or computer, which is communicatively coupled to the server. The one or more processors may be configured to send one or more of the first, second, or third risk values 616, and/or the risk assessment 708 to the display system, to notify a user of a risk value. The system 700 may alternatively or further comprise an auditory notification system, such that a noise associated with a risk value 616 or risk assessment 708 notifies a user of the risk value 616.

The risk assessment 708 may be in the form of “high”, “low”, “medium”, for example to notify of a risk of post-operative leakage. It may be in the form of a percentage. It may be sent to a patient, their healthcare provider, or other designated users.

The risk assessment 708 may comprise error and/or confidence intervals, generated by statistics associated with the various models and the quantity and quality of data in the database 706.

The risk assessment 708 may be used to inform a post-operative treatment plan, and/or automatically update a post-operative treatment plan, for the target patient 710.

The combined risk assessment model 610 may be trained on one or more risk assessment models. The historical data risk assessment model 608 may in turn be trained based off of various types of historical data, including, but not limited to, pre-operative risk factors such as ASA scores, surgical approach, patient medical records (i.e., EMRs), pre-operative physical conditions, and the like. The real-time data risk assessment model 604 may be trained off of various types of time-marked data 716, including biosensor data and the like.

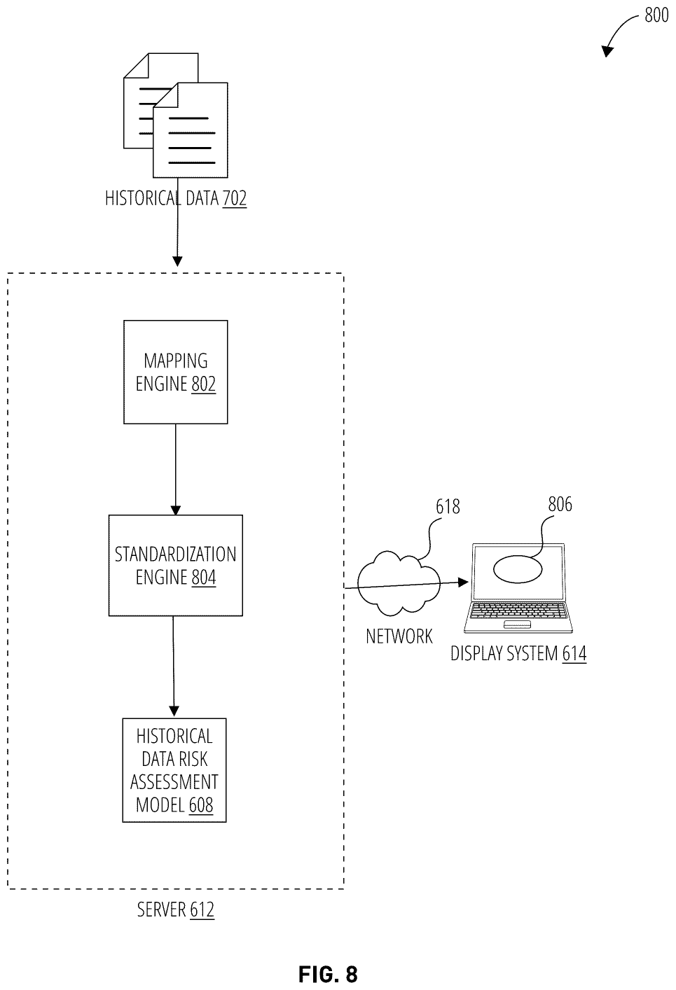

FIG. 8 illustrates a schematic demonstrating a method for training a historical data risk assessment model 800 using historical data 702.

The method comprises sending, for a plurality of patients, historical data and an indication of whether the patient associated with the historical data suffered a complication, to a server 612, pre-processing the historical data 702 by mapping the historical data to numerical values with a mapping engine 802, standardizing the data with a standardization engine 804, and inputting the mapped and standardized data into the historical data risk assessment model 608. The model 608 comprises one or more regression models which are trained by associating, for a plurality of patients, a given patients' pre-processed historical data with the indication of whether the given patient suffered a complication.

In the example of an anastomotic leak risk assessment, training the historical data risk assessment model 608 may comprise:

Preprocessing of historical data, via the mapping engine 802 and standardization engine 804:
1) Collecting historical data 702 from a plurality of patients—in this case, the historical data 702 comprising pre-operative risk factors, such as an ASA Score and surgical approach (laparotomy, laparoscopy, robotic), and an indication of whether the patient associated with the historical data 702 suffered a complication, for each patient in the available training data.
2) Mapping, via the mapping engine 802, ASA Score to numerical values (for example, ASA I: 1, ASA II: 2, ASA III: 3, ASA IV: 4, ASA V: 5) and surgical approach to numerical values (for example, laparoscopy and robotic: 0, laparotomy: 1), and standardize, via the standardization engine 804 the set of training values to have a mean of 0 and a standard deviation of 1.
Training of historical data risk assessment model 608:
3) Training one or more regression models with the numerical values and associated indication of a complication. For example, a Logistic Regression model (using an L2 penalty and balanced class weights), may be trained to predict a risk value 616 in the form of a probability of the patient to have an Anastomotic Leak given the numerical surgical approach and ASA score.

The above is an example of the historical data risk assessment model 608 being trained, using pre-operative data, to give a pre-operative risk-factor historical data risk values 806.

The historical data risk assessment model 608 may also be trained using patients' individual health indicators, such as gender, height, weight, or past surgeries, past surgical complications, surgical parameters (including, but not limited to, duration, blood loss, etc.) and vitals data (heart rate, respiratory rate, oxygen saturation, etc.).

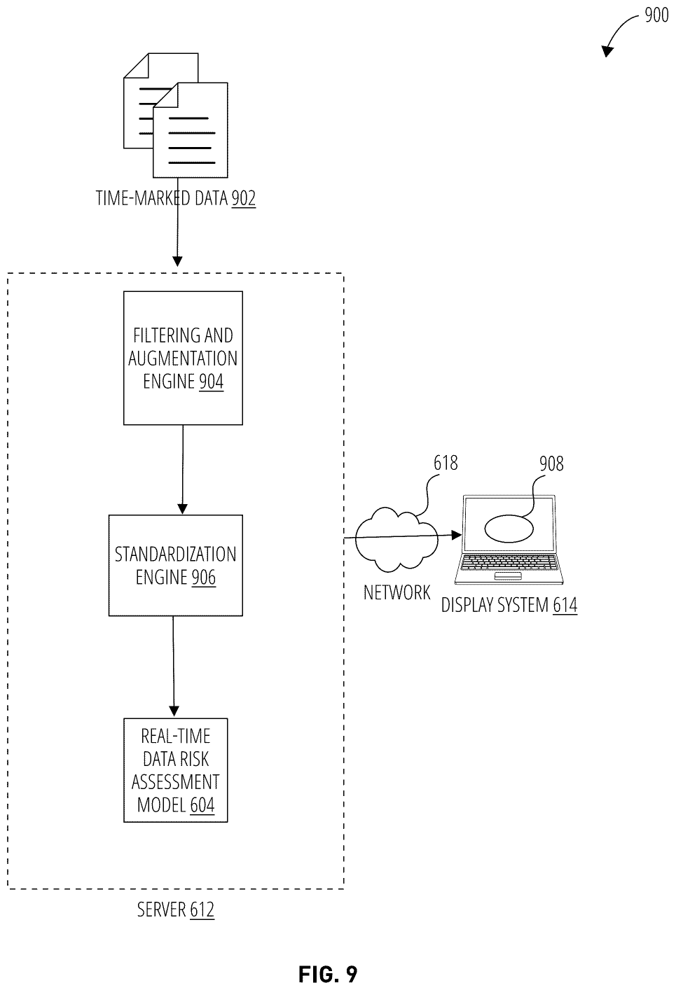

FIG. 9 illustrates a schematic demonstrating a method for training a real-time data risk assessment model 604 using time-marked data 716

The method comprises sending, for a plurality of patients, time-marked data 902, generally by receiving signals from sensors coupled with the patients, and an indication of whether the patient associated with the time-marked data 716 suffered a complication, a server, pre-processing the time-marked data 716, which may comprise different techniques of filtering and augmenting signals received from sensors, depending on the nature of the time-marked data 716, via a filtering and augmentation engine 904, standardizing the data with a standardization engine 906, and inputting the filtered, augmented, and standardized data into the real-time data risk assessment model 604. Filtering the data into time-based means may comprise decimating sensor measurements to larger/smaller means (e.g., 30 minutes) or aggregating using a statistic such as median/mean, etc.

The model 604 comprises one or more regression models which are trained by associating, for a plurality of patients, a given patients' time-marked data with the indication of whether the given patient suffered a complication. When a target patient is assessed in real-time, the time post-surgery, as well as real-time sensor measurements, may be inputted into the real-time data risk assessment model 604 to estimate a real-time data risk value 908 of whether the target patient may suffer a complication at the given post-operative time which may be displayed on the display system 614.

In the example of an anastomotic leak risk assessment, training the real-time data risk assessment model 604 may comprise:

Preprocessing of pH data, via the filtering and augmentation engine 904:
1) Removing erroneous sensor measurements with pH and EC measurements (from continuous measurements from an inline device 622, in fluid communication with a patient, out of expected bounds, as well as any measurements that are from calibration fluids).
2) For patients who experienced an Anastomotic Leak, removing all pH measurements after their diagnosis (this is only performed on patients used to train the model).
3) Decimating the filtered pH signal to 15-minute means. Depending on desired resolution of the data, this time frame may be smaller or larger.
Standardizing pH data, via the standardization engine 804:
4) For a given post-operative hour, calculating the mean pH sensor reading for the 24 hours prior to the post-operative hour and standardizing the calculated means from the training set to have a mean of 0 and a standard deviation of 1 (if making a prediction on a new patient, use the standardization values calculated on the training data).
Training the real-time data risk assessment model 604:
5) Using the standardized mean pH sensor readings for each patient in the available training data, training one or more regression models, such as Logistic Regression models (using an L2 penalty and balanced class weights) to predict a risk value 616 in the form of a probability of the patient to have an Anastomotic Leak given their mean pH for that post-operative hour. In some embodiments, a plurality of models may be trained for specific time intervals, such as every hour following surgery where predictions of a risk value are made.

The above is an example of the real-time data risk assessment model 604 being trained, using continuous sensor measurements relating to the real-time condition of the patient, to continuously estimate a post-operative risk-factor risk value 616.

The real-time data risk assessment model 604 could also be trained using data from different sensors that measure in real-time or as point-measurements (e.g., Blood Gas Analyzers, etc.), quantitative and qualitative data obtained by a healthcare professional (e.g., heart rate, heart rate variability, blood pressure, SpO2, temperature, ECG, etc.), colour monitoring of patient fluids (blood, urine, bile etc.), or any other relevant physiological data relating to the condition of the patient.

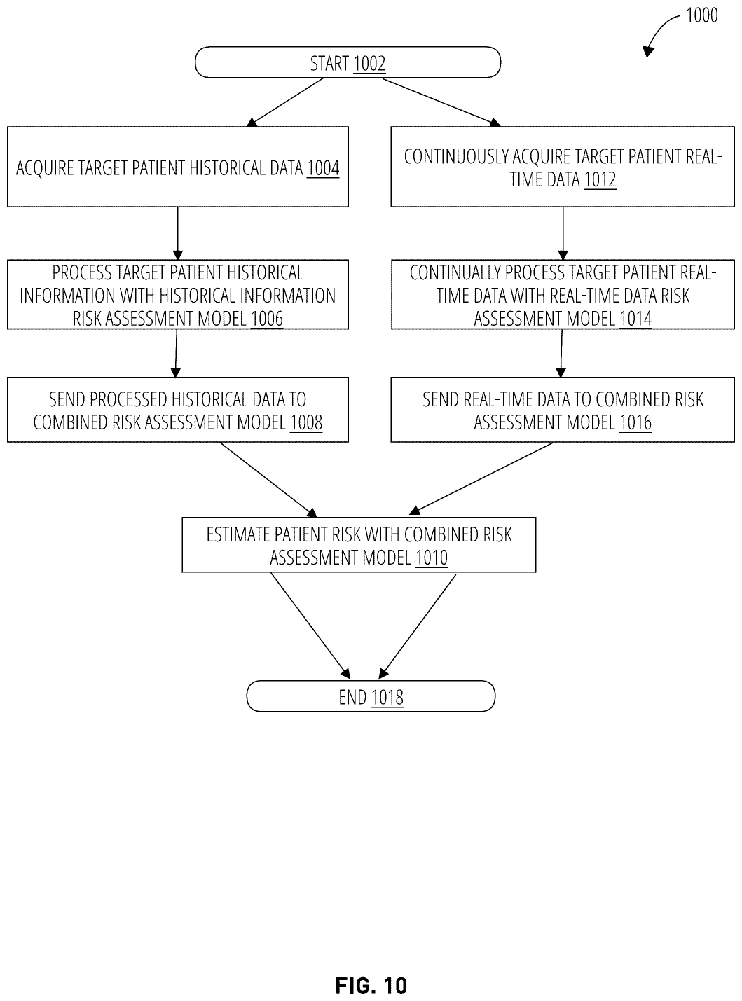

FIG. 10 is an exemplary process flow diagram for a method for estimating a risk value of a patient 1000.

The method 1000 starts at step 1002.

At step 1004, the system acquires historical data from the target patient. At step 1012, the system begins to continuously acquire real-time data from the patient. Generally, steps 1004 and 1012 are run in parallel, however they may be run sequentially, or in the case of patients where one form of data may not be available, only one of these steps may be run instead.

At step 1006, the historical information is pre-processed and processed using the historical data risk assessment model 608. This step generally involves estimating, with the risk model 608 a risk value from the processed historical data. At step 1014, the real-time data is continuously pre-processed and processed using the real-time data risk assessment model 604. This step generally involves estimating, with the risk model 604, a risk value from the real-time data. Generally, steps 1006 and 1014 are run in parallel, however they may be run sequentially, or in the case of patients where one form of data may not be available, only one of these steps may be run instead.

At step 1008, the processed historical data or estimated risk value from step 1006, is sent to the combined risk assessment model. In parallel or separately, in step 1016, the processed real-time data or estimated risk value from step 1014, is sent to the combined risk assessment model.

At step 1010, the combined risk assessment model uses the risk values sent from the separate models in steps 1008 and 1016, to estimate a risk assessment for the patient.

At step 1018, the method ends.

The method 1000 may further comprise continuously assessing risk, i.e., continuously repeating steps 512-1010 with continuously generated real-time data.

It may further comprise notifying or displaying a risk assessment, in the form of a visual, auditory, or haptic notification.

In some embodiments, different machine-learning algorithms or techniques may be used, alone or in combination, in the different engines noted above.

These may include, for example, deep learning architectures such as Deep Belief Network (DBN), Stacked Auto Encoder (SAE), Convolutional Neural Network (CNN) or Recurrent Neural Network (RNN) may be used. Other examples include, without limitation, Restricted Boltzmann machines (RBM), Social Restricted Boltzmann Machines (SRBM), Fuzzy Restricted Boltzmann Machines (FRBM), TTRBM models of Deep Belief Networks (DBN) or similar approaches could be used; AE, FAE, GAE, DAE, BAE models of Statistically Adjusted End Use (SAE) models could be used; models such as AlexNet, ResNet, Inception, VGG16, ECNN models of CNN may be used; Bidirectional Recurrent Neural Networks (BiRNN), Long Short-Term Memory (LSTM) networks, Gate Recurrent Unit (GRU) of RNN may also be used. Additional techniques specific to time-series modelling may be used, including, but not limited to, dynamic time warping, change point detection, and ARIMA.

In some embodiments, other types of algorithms such as physics-based mathematical computations and basic multiple linear regression models may also be relied upon in conjunction with or in complementarity with those architectures and learning algorithms.

Many of the functional units described in this specification have been labeled as “engines”, in order to more particularly emphasize their implementation independence. For example, an engine may be implemented as a hardware circuit comprising custom VLSI circuits or gate arrays, off-the-shelf semiconductors such as logic chips, transistors, or other discrete components. An engine may also be implemented in programmable hardware devices such as field programmable gate arrays, programmable array logic, programmable logic devices or the like.

Engines may also be implemented in software for execution by various types of processors. An identified engine of executable code may, for instance, comprise one or more physical or logical blocks of computer instructions which may, for instance, be organized as an object, procedure, or function. Nevertheless, the executables of an identified engine need not be physically located together, but may comprise disparate instructions stored in different locations which, when joined logically together, comprise the engine and achieve the stated purpose for the module.

Indeed, an engine of executable code may be a single instruction, or many instructions, and may even be distributed over several different code segments, among different programs, and across several memory devices. Similarly, operational data may be identified and illustrated herein within engines, and may be embodied in any suitable form and organized within any suitable type of data structure. The operational data may be collected as a single data set, or may be distributed over different locations including over different storage devices, and may exist, at least partially, merely as electronic signals on a system or network. Where an engine or portions of an engine are implemented in software, the software portions are stored on one or more computer readable storage media.

It should be understood that the models described herein may be trained using various sources of data.

Additional models may ensembled along with the pre-operative risk factor and pH model (for example, a vitals model), to be used in conjunction with, or as an alternative to, models described above.

More sophisticated individual models may be developed, for use in conjunction with, or as an alternative to, the models described above. For example, a pre-operative risk factor model that also takes volume of blood lost during surgery, or one that uses a Random Forest Model to predict a probability.

Filtering the data into time-based means may comprise decimating sensor measurements to larger/smaller means (e.g., 30 minutes) or aggregating using a statistic such as median/mean, etc.

Alternative methods of ensembling predictions from individual models may be employed (for example, passing into a Logistic Regression model instead of using thresholds for classification).

Systems and methods described herein may further comprise features such as a prompt to request inputs of additional data at key times to update model. For example, a prompt for HR at post-operative hour 6, which can be used to update a risk assessment.

The combined risk assessment model or algorithm can be deployed in firmware or cloud and updated/improved as needed. It can be further adapted to detect different types of post operative complications.

The combined risk assessment model may be used with an inline device, continuously receiving data, in real-time, from biosensors or sensors in fluid communication from a patient, or adapted to work with other sensor systems, or be adapted to receive manual inputs of data.

The present disclosure includes systems having processors to provide various functionality to process information, and to determine results based on inputs. Generally, the processing may be achieved with a combination of hardware and software elements. The hardware aspects may include combinations of operatively coupled hardware components including microprocessors, logical circuitry, communication/networking ports, digital filters, memory, or logical circuitry. The processors may be adapted to perform operations specified by a computer-executable code, which may be stored on a computer readable medium.

The steps of the methods described herein may be achieved via an appropriate programmable processing device or an on-board field programmable gate array (FPGA) or digital signal processor (DSP), that executes software, or stored instructions. In general, physical processors and/or machines employed by embodiments of the present disclosure for any processing or evaluation may include one or more networked or non-networked general purpose computer systems, microprocessors, field programmable gate arrays (FPGA's), digital signal processors (DSP's), micro-controllers, and the like, programmed according to the teachings of the exemplary embodiments discussed above and appreciated by those skilled in the computer and software arts. Appropriate software can be readily prepared by programmers of ordinary skill based on the teachings of the exemplary embodiments, as is appreciated by those skilled in the software arts. In addition, the devices and subsystems of the exemplary embodiments can be implemented by the preparation of application-specific integrated circuits, as is appreciated by those skilled in the electrical arts. Thus, the exemplary embodiments are not limited to any specific combination of hardware circuitry and/or software.

Stored on any one or a combination of computer readable media, the exemplary embodiments of the present invention may include software for controlling the devices and subsystems of the exemplary embodiments, for processing data and signals, for enabling the devices and subsystems of the exemplary embodiments to interact with a human user or the like. Such software can include, but is not limited to, device drivers, firmware, operating systems, development tools, applications software, and the like. Such computer-readable media further can include the computer program product of an embodiment of the present invention for preforming all or a portion (if processing is distributed) of the processing performed in implementations. Computer code devices of the exemplary embodiments of the present invention can include any suitable interpretable or executable code mechanism, including but not limited to scripts, interpretable programs, dynamic link libraries (DLLs), complete executable programs and the like.

Common forms of computer-readable media may include, for example, magnetic disks, flash memory, RAM, a PROM, an EPROM, a FLASH-EPROM, or any other suitable memory chip or medium from which a computer or processor can read.

While particular implementations and applications of the present disclosure have been illustrated and described, it is to be understood that the present disclosure is not limited to the precise construction and compositions disclosed herein and that various modifications, changes, and variations can be apparent from the foregoing descriptions without departing from the spirit and scope of the present disclosure.

What is claimed is:

1. A computer-implemented method for assessing a risk value of a target patient, the method comprising:
receiving at a server, via a connection mechanism, target patient data;
estimating at the server, using one or more risk assessment models, one or more risk values associated with the target patient data.

2. The computer-implemented method of claim 1, wherein said target patient data comprises one or more of: historical data, patient population-level data, and real-time data.

3. The computer-implemented method of claim 2, wherein the one or more risk assessment models comprise one or more of: a historical data risk assessment model, a real-time data risk assessment model, and a combined risk assessment model, the combined risk assessment model comprising one or more risk assessment models.

4. The computer-implemented method of claim 3, wherein the historical data risk assessment model is trained by:
receiving, at a server comprising one or more processors and a memory, historical data corresponding to a plurality of patients, the one or more processors comprising one or more of a mapping engine and a standardization engine, the historical data for each patient of the plurality of patients corresponding to an indication of whether a patient encountered a complication;
generating pre-processed historical data by:
mapping, via the mapping engine, the historical data for each patient of the plurality of patients, to numerical values;
standardizing, via the standardization engine, historical data for each patient of the plurality of patients;
performing a regression analysis on the pre-processed data, the regression analysis determining a relationship between historical data and a risk value.

5. The computer-implemented method of claim 3, wherein the real-time data risk assessment model is trained by:
receiving, at a server comprising one or more processors and a memory, time-marked data corresponding to signal data measured by a plurality of sensors coupled to a plurality of patients, the one or more processors comprising one or more of a filtering and augmentation engine and a standardization engine, the time-marked data for each patient of the plurality of patients corresponding to an indication of whether a patient encountered a complication;
generating pre-processed time-marked data by:
filtering and augmenting, via the filtering and augmentation engine, the time-marked data for each patient of the plurality of patients;
standardizing, via the standardization engine, the time-marked data for each patient of the plurality of patients;
performing one or more regression analyses on the pre-processed time-marked data, the one or more regression analyses determining one or more relationships between real-time data and a risk value.

6. The computer-implemented method of claim 2, wherein the historical data comprises one or more of the target patient's, or target patient population's: pre-operative risk factors, medical records, surgical history, individual health indicators, and surgical parameters.

7. The computer-implemented method of claim 2, wherein the real-time data comprises sensor data from one or more sensors continuously measuring signals associated with a physiological condition of the target patient.

8. The computer-implemented method of claim 1, further comprising notifying a user of said risk value.

9. The computer-implemented method of claim 7, wherein the risk value is estimated continuously.

10. The computer-implemented method of claim 1, wherein the risk value comprises a probability of the target patient developing a post-surgical complication.

11. A system for assessing a risk value for a target patient, the system comprising:
a server, one or more processors, and a memory, the one or more processors communicatively coupled to a database, the database comprising historical data and time-marked data from a plurality of patients;
the one or more processors configured to receive, via a connection mechanism, target patient data; and,
the memory comprising instructions, that, when executed by the one or more processors, configures the server to:
receive, at the server, said target patient data;
pre-process or process, via a processor, said target patient data;
estimate, using one or more risk assessment models, one or more risk values associated with the target patient data.

12. The system of claim 11, wherein said target patient data comprises one or more of: historical data, patient population-level data, and real-time data.

13. The system of claim 11, wherein the one or more risk assessment models comprise one or more of: a historical data risk assessment model, a real-time data risk assessment model, and a combined risk assessment model, the combined risk assessment model comprising one or more risk assessment models.

14. The system of claim 13, wherein the historical data risk assessment model is trained by:
receiving, at a server comprising one or more processors and a memory, historical data corresponding to a plurality of patients, the one or more processors comprising one or more of a mapping engine and a standardization engine, the historical data for each patient of the plurality of patients corresponding to an indication of whether a patient encountered a complication;
generating pre-processed historical data by:
mapping, via the mapping engine, the historical data for each patient of the plurality of patients, to numerical values;
standardizing, via the standardization engine, historical data for each patient of the plurality of patients;
performing a regression analysis on the pre-processed data, the regression analysis determining a relationship between historical data and a risk value.

15. The system of claim 13, wherein the real-time data risk assessment model is trained by:
receiving, at a server comprising one or more processors and a memory, time-marked data corresponding to signal data measured by a plurality of sensors coupled to a plurality of patients, the one or more processors comprising one or more of a filtering and augmentation engine and a standardization engine, the time-marked data for each patient of the plurality of patients corresponding to an indication of whether a patient encountered a complication;
generating pre-processed time-marked data by:
filtering and augmenting, via the filtering and augmentation engine, the time-marked data for each patient of the plurality of patients;
standardizing, via the standardization engine, the time-marked data for each patient of the plurality of patients;
performing one or more regression analyses on the pre-processed time-marked data, the one or more regression analyses determining one or more relationships between real-time data and a risk value.

16. The system of claim 11, wherein the risk value comprises a probability of the target patient developing a post-surgical complication.

17. The system of claim 11, further comprising a display system for displaying the risk value.

18. A non-transitory computer-readable storage medium, the computer-readable storage medium including instructions that when executed by a computer, cause the computer to the steps of:
receive, via a connection mechanism, target patient data;
pre-process or process, via a processor, said target patient data;
estimate, using one or more risk assessment models, one or more risk values associated with the target patient data.

19. The non-transitory computer-readable storage medium of claim 18, wherein said target patient data comprises one or more of: historical data, patient population-level data, and real-time data.
